(19) United States
(12) Patent Application Publication
(10) Pub. No.: US 2015/0337349 A1
(43) Pub. Date: Nov. 26, 2015

(54) Microbiome Modulation Index

(71) Applicant: SECOND GENOME, INC., South San Francisco, CA (US)

(72) Inventors: Justin Kuczynski, San Francisco, CA (US); Mohan Iyer, Menlo Park, CA (US); Nadir Mahmood, San Francisco, CA (US); Rachel Steger, Sunnyvale, CA (US); Peter DiStefano, Southborough, MA (US)

(73) Assignee: Second Genome, Inc., South San Francisco, CA (US)

(21) Appl. No.: 14/758,934

(22) PCT Filed: Jan. 3, 2014
(86) PCT No.: PCT/US14/10244

§ 371 Date: Jul. 1, 2015

Related U.S. Application Data
(60) Provisional application No. 61/749,229, filed on Jan. 4, 2013.

(51) Int. Cl. C12Q 1/02; G06F 19/00; C12Q 1/68

(52) U.S. Cl. CPC C12Q 1/025; C12Q 1/689; G06F 19/702; G06F 19/3431; C12Q 2600/136

(57) ABSTRACT

The disclosure provides methods and systems for characterizing the effects of an agent on one or more microbial communities.

### CROSS-REFERENCE

This application claims the benefit of U.S. Provisional Patent Application No. 61/749,229, filed Jan. 4, 2013, which is incorporated herein by reference in its entirety for all purposes.

### BACKGROUND

Various microbiota found in a living organism provide many crucial contributions to its host, including, for example, aiding digestion, aiding in the development of immune systems, and/or imparting resistance to pathogenic colonization. Even a slight fluctuation in the symbiotic balance between microbiota and its host may be deleterious to the host, possibly leading to a pathological condition. For example, perturbations in the human gut may lead to conditions such as Clostridium difficile infection or inflammatory bowel disease (IBD). The composition of a microbial community can undergo changes as a result of interactions between the microbiota and a host's immune and metabolic systems, and/or interactions between the microbiota and exogenous agents. In one example, human exposure to antibiotics is known to have both short-term and long-term effects on the composition of various host microbiota, including those of the gut.

### SUMMARY

The disclosure provides method and systems for evaluating changes to microbial communities when contacted with one or more agents. In some aspects, the disclosure provides methods and systems for determining a quantitative measure of such effects. In some embodiments, the quantitative measure is an index, such as, for example, a Microbiome Modulation Index (MMI).

An aspect of the disclosure provides a method for characterizing an agent comprising: (a) enumerating abundance of one or more microbial taxa or related chemical species in one or more first samples obtained from a first subject prior to administering the agent to the subject; (b) administering the agent to the first subject; (c) enumerating abundance of the one or more microbial taxa or related chemical species in one or more second samples obtained from the first subject after the administering the agent to the first subject; (d) generating an index for the agent using: (i) the enumerating abundance of the one or more microbial taxa or related chemical species in one or more first samples obtained from a first subject prior to administering the agent to the subject in step (a); (ii) the enumerating abundance of the one or more microbial taxa or related chemical species in one or more second samples obtained from the first subject after the administering the agent to the first subject in step (b); and (iii) at least one of a prevalence weight, a variability weight, or a condition importance weight.

In some embodiments, the method further comprises comparing the index with one or more reference indices, wherein the comparing is used to make a health decision with respect to the agent. In some embodiments, the variability weight is generated using the relative abundance variability of each of the one or more microbial taxa or related chemical species in samples obtained from the first subject, wherein the agent has not been administered to the first subject. In some embodiments, the prevalence weight is calculated using the relative abundance of each of the microbial taxa or related chemical species in third samples obtained from a second subject. In some embodiments, the first subject and the second subject are of a different species. In some embodiments, the first subject is a species of mouse and the second subject is a human.

In some embodiments, the condition importance weight is with respect to a condition of interest selected from the group consisting of Clostridium difficile infection, inflammatory bowel disease (IBD), a condition of the gut, Crohn's Disease (CD), irritable bowel syndrome (IBS), stomach ulcers, colitis, neonatal necrotizing enterocolitis, gastroesophageal reflux disease (GERD), gastroparesis, cystic fibrosis, chronic obstructive pulmonary disease, rhinitis, atopy, asthma, acne, a food allergy, obesity, periodontal disease, diarrhea, constipation, functional bloating, gastritis, lactose intolerance, visceral hyperalgesia, colic, pouchitis, diverticulitis, allergies, asthma, sinusitis, chronic obstructive pulmonary disorder (COPD), depression, attention deficit hyperactivity disorder (ADHD), autism, Alzheimers, migraines, multiple sclerosis (MS), Lupus, arthritis, Type 2 diabetes, obesity, non alcoholic steato hepatitis (NASH), non alcoholic fatty liver disease (NAFLD), risk of infarction/cardiovascular risk, heart failure, cancer, dental caries, gingivitis, oral cancer, oral mucositis, bacterial vaginosis, fertility, sinusitis, allergies, cystic fibrosis, lung cancer, psoriasis, atopic dermatitis, methicillin-resistant staphylococcus aureus (MRSA), and combinations thereof.

In some embodiments, a plurality of first subjects are administered the agent, wherein the one or more first samples are obtained from each first subject of the plurality prior to the administering, wherein the one or more second samples are obtained from each first subject of the plurality after the administering, and wherein the index is calculated using the equation:

index= d̅/d₀
wherein d̅ is the average value d calculated for each first subject in the plurality,
wherein d is calculated using the equation:

$$d = \frac{{Σ}_{i} {g}_{i} * {f}_{i} * {h}_{i} * \vert {A}_{Ti} - {A}_{0 i} \vert}{{Σ}_{i} {A}_{1 i} + {A}_{0 i}}$$

wherein gᵢ is the variability weight,
wherein fᵢ is the prevalence weight,
wherein hᵢ is the condition importance weight,
wherein A_{Ti} is the abundance of the microbial taxa or related chemical species i in the one or more second samples obtained at time T after the administering, and
wherein A₀ᵢ is the abundance of the microbial taxa or related chemical species i in the one or more first samples obtained prior to the administering; and

wherein d₀ is the average value d calculated for a plurality of third samples obtained from one or more second subjects identical to the first subjects but not administered the agent.

In some embodiments, the first subject is a type of living organism selected from the group consisting of a mammal, a rodent, a mouse, a rat, a dog, a cat, a hamster, a monkey, a pig, a squirrel, a guinea pig, a gerbil, a bird, a hydra, a rabbit, a fish, a frog, a cow, a lobster, a lamb, a chicken, a Drosphilia, a Xenopus, a livestock, a companion animal, and a human. In some embodiments, the first subject is a type of living organism that is a genetically-modified species or a gnotobiotic species. In some embodiments, the first subject is an in vitro culture of one or more microbes.

In some embodiments, the health decision is made for at least one second subject that is of a different type of living organism than the first subject. In some embodiments, the number of first subjects is at least 3 first subjects, at least 10 first subjects, at least 30 first subjects, or at least 50 first subjects. In some embodiments, the first subject is a species of a mouse and the second subject is a human.

In some embodiments, the number of the microbial taxa or related chemical species is at least 10 microbial taxa or related chemical species, at least 100 microbial taxa or related chemical species, at least 1000 microbial taxa or related chemical species, at least 10000 microbial taxa or related chemical species, at least 100,000 microbial taxa or related chemical species, or at least 1,000,000 microbial taxa or related chemical species. In some embodiments, the microbial taxa are operational taxonomic units (OTUs). In some embodiments, the OTUs are formed by clustering nucleic acid sequences of microbial organisms based on gene sequence homology. In some embodiments, the OTUs are characterized by microbes having at least 80%, at least 85%, at least 90%, or at least 95% 16S RNA sequence homology. In some embodiments, the microbial taxa are selected from the group consisting of domains, kingdoms, phylums, classes, orders, families, genera, and single species.

In some embodiments, the one or more first samples and the one or more second samples are obtained from at least one of the following: the gut, the vagina, the cervix, the respiratory system, the ear, nasal passages, an oral cavity, a sinus, a nare, the urogenital tract, skin, feces, udders, auditory canal, earwax, breast milk, blood, sputum, urine, saliva, open wounds, secretions from open wounds, and combinations thereof. In some embodiments, the one or more first samples and the one or more second samples are obtained from feces.

In some embodiments, the agent is selected from the group consisting of a microbe, a virus, a prebiotic, a probiotic, a synbiotic, a fecal transplant, a small molecule drug, a biologic drug, an orally administered drug, a parenterally administered drug, an antibiotic, a food, a beverage, a nutraceutical, a supplement, a beauty care product, personal hygiene product, an allergen, a household chemical, a wound dressing, a wound antiseptic, an industrial chemical, a hazardous chemical, water from a municipal water source, an environmental sample, an aerosol that may be inhaled via the nose or throat, a topical pain reliever, a material used to make clothing, and combinations thereof. In some embodiments, the index is generated for the agent with respect to a second subject that is of a different type of living organism than the first subject.

In some embodiments, the health decision is determining the safety and/or efficacy of the agent. In some embodiments, two or more agents are administered in step (b) and the health decision is determining the safety and/or efficacy of administering the two or more agents in combination. In some embodiments, the health decision is deciding whether the agent can ameliorate the deleterious effects of one or more other agents on the one or more microbial taxa or related chemical species. In some embodiments, the health decision is deciding whether to develop the agent into a drug. In some embodiments, the health decision is made prior to or during a clinical trial of the agent.

In some embodiments, the agent is a drug. In some embodiments, the drug is selected from the group consisting of Prozac, Precose, Ambien, Mesalamine, Nexium, Seroquel, Cymbalta, Crestor, Lipitor, Plavix, Actos, glucophage, Belviq, Qsymia, estrogen, a synthroid, lisinopril, lotensin, azithromycin, amoxicillin, Pentasa, Ritalin, Viagra, Diflucan, Prilosec, ibuprofen, aspirin, Ensure, Slim Fast, PediaSure, Claritin, Benadryl, caffeine, and combinations thereof. In some embodiments, the agent is a drug and the health decision is determining the safety and/or efficacy of the drug to treat a condition. In some embodiments, the agent is a drug and the health decision is determining a dose of the drug.

In some embodiments, the agent is a food, optionally a food of a diet. In some embodiments, the health decision is determining whether the agent causes a condition. In some embodiments, the condition is selected from the group consisting of Clostridium difficile infection, inflammatory bowel disease (IBD), a condition of the gut, Crohn's Disease (CD), irritable bowel syndrome (IBS), stomach ulcers, colitis, neonatal necrotizing enterocolitis, gastroesophageal reflux disease (GERD), gastroparesis, cystic fibrosis, chronic obstructive pulmonary disease, rhinitis, atopy, asthma, acne, a food allergy, obesity, periodontal disease, diarrhea, constipation, functional bloating, gastritis, lactose intolerance, visceral hyperalgesia, colic, pouchitis, diverticulitis, allergies, asthma, sinusitis, chronic obstructive pulmonary disorder (COPD), depression, attention deficit hyperactivity disorder (ADHD), autism, Alzheimers, migraines, multiple sclerosis (MS), Lupus, arthritis, Type 2 diabetes, obesity, non alcoholic steato hepatitis (NASH), non alcoholic fatty liver disease (NAFLD), risk of infarction/cardiovascular risk, heart failure, cancer, dental caries, gingivitis, oral cancer, oral mucositis, bacterial vaginosis, fertility, sinusitis, allergies, cystic fibrosis, lung cancer, psoriasis, atopic dermatitis, methicillin-resistant staphylococcus aureus (MRSA), and combinations thereof.

In some embodiments, the enumerating the abundance of the one or more microbial taxa or related chemical species in step (a) and step (c) is completed by detecting a species selected from the group consisting of a nucleic acid, a lipid, a carbohydrate, a protein, a peptide, a small molecule, and combinations thereof. In some embodiments, the enumerating the abundance of the one or more microbial taxa or related chemical species is completed by detecting a nucleic acid. In some embodiments, the nucleic acid is all or a portion of a 16S ribosomal RNA (rRNA) gene or the 16S rRNA product of the gene. In some embodiments, one or more of steps (a)-(d) are completed with the aid of a processor. In some embodiments, one or more of steps (a)-(d) are completed using the internet. In some embodiments, the index is transmitted or received over the internet.

In another aspect, the disclosure provides a method of providing health counseling, comprising: (a) identifying a subject in want or need of an agent; (b) characterizing the agent by generating an index for the agent; and (c) providing counseling regarding the agent to the subject using the index. In some embodiments, the agent is characterized by a method comprising (a) enumerating abundance of one or more microbial taxa or related chemical species in one or more first samples obtained from a first subject prior to administering the agent to the subject; (b) administering the agent to the first subject; (c) enumerating abundance of the one or more microbial taxa or related chemical species in one or more second samples obtained from the first subject after the administering the agent to the first subject; (d) generating an index for the agent using: (i) the enumerating abundance of the one or more microbial taxa or related chemical species in one or more first samples obtained from a first subject prior to administering the agent to the subject in step (a); (ii) the enumerating abundance of the one or more microbial taxa or related chemical species in one or more second samples obtained from the first subject after the administering the agent to the first subject in step (b); and (iii) at least one of a prevalence weight, a variability weight, or a condition importance weight.

In some embodiments, the first subject used to generate the index is the same type of living organism as the subject in want or need of the agent. In some embodiments, the first subject used to generate the index is not the same type of living organism as the subject in want or need of the agent. In some embodiments, the first subject used to generate the index is a mouse and the subject in want or need of the agent is a human.

In some embodiments, the agent is selected from the group consisting of a microbe, a related chemical species to a microbe, a virus, a prebiotic, a probiotic, a synbiotic, a fecal transplant, a small molecule drug, a biologic drug, an orally administered drug, a parenterally administered drug, an antibiotic, a food, a beverage, a nutraceutical, a supplement, a beauty care product, personal hygiene product, an allergen, a household chemical, a wound dressing, a wound antiseptic, an industrial chemical, a hazardous chemical, water from a municipal water source, an environmental sample, an aerosol that may be inhaled via the nose or throat, a topical pain reliever, a material used to make clothing, and combinations thereof. In some embodiments, the counseling is with respect to a plurality of agents. In some embodiments, the plurality of agents is a combination therapy or a diet.

In some embodiments, the counseling includes the generation of a report and/or providing the report to the subject. In some embodiments, the counseling includes any of the following: providing the subject with information regarding the efficacy of the agent; providing the subject with information regarding the safety of the agent; providing the subject with information regarding the safety of the agent when administered with one or more different agents; providing the subject with information regarding the efficacy of the agent when administered with one or more different agents; providing the subject with a recommendation to use or continue to use the agent or a combination of agents including the agent; providing the subject with a recommendation to not use or discontinue use of the agent or a combination of agents comprising the agent; providing the subject with a ranked list including the agent or a combination of agents comprising the agent for use or continued use; providing the subject with a recommendation for the addition of the agent to a regimen comprising one or more different agents; providing the subject with a recommendation for monitoring use of the agent over time; providing the subject with a recommended dose of the agent or a combination of agents comprising the agent; and combinations thereof. In some embodiments, the counseling is provided to the subject by a health care professional.

In another aspect, the disclosure provides a specialized computer system that is capable of performing the following: (a) accepting raw data that can be used to enumerate microbial taxa or related chemical species and characterize an agent by generating an index for the agent; (b) processing the raw data such that it may be used to calculate the index; (c) calculating the index; and (d) outputting the index to a user. In some embodiments, the agent is characterized by a method comprising (a) enumerating abundance of one or more microbial taxa or related chemical species in one or more first samples obtained from a first subject prior to administering the agent to the subject; (b) administering the agent to the first subject; (c) enumerating abundance of the one or more microbial taxa or related chemical species in one or more second samples obtained from the first subject after the administering the agent to the first subject; (d) generating an index for the agent using: (i) the enumerating abundance of the one or more microbial taxa or related chemical species in one or more first samples obtained from a first subject prior to administering the agent to the subject in step (a); (ii) the enumerating abundance of the one or more microbial taxa or related chemical species in one or more second samples obtained from the first subject after the administering the agent to the first subject in step (b); and (iii) at least one of a prevalence weight, a variability weight, or a condition importance weight.

In some embodiments, the specialized computer system is capable of generating a report. In some embodiments, the report is used for the outputting the index to the user. In some embodiments, the outputting is via an electronic display of the specialized computer system or via a printer of the specialized computer system. In some embodiments, the electronic display comprises a graphical user interface (GUI). In some embodiments, the specialized computer system is capable of aiding in making one or more health decisions with respect to the agent and/or aiding in providing counseling to a subject in want or need of the agent. In some embodiments, the specialized computer system comprises any of the following databases: a database comprising reference indices, a database comprising nucleic acid sequences, a database comprising prevalence weights, a database comprising variability weights, a database of calculated indices, a database comprising microbial taxa classification schemes, a database comprising microbial taxa and/or related chemical species, and combinations thereof. In some embodiments, the specialized computer system is capable of transmitting or receiving data over a computer network. In some embodiments, the computer network is the internet.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an exemplary computer system for executing methods of the disclosure.

FIG. 2 is a flowchart summarizing experiments described in Example 2.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” are intended to include the plural forms as well, unless the context clearly indicates otherwise. Furthermore, to the extent that the terms “including”, “includes”, “having”, “has”, “with”, “such as”, or variants thereof, are used in either the specification and/or the claims, such terms are not limiting and are intended to be inclusive in a manner similar to the term “comprising”.

The term “about,” as used herein, generally refers to a range that is 15% greater than or less than a stated numerical value within the context of the particular usage. For example, “about 10” would include a range from 8.5 to 11.5.

The term “microbiome,” as used herein, generally refers to the totality, or a subset of the totality, of microbes, their genetic elements (genomes), and interactions with a particular environment. Such an environment, for example, may be a region of a living organism.

The term “microbiota,” as used herein, generally refers to the microflora and/or microfauna in an ecosystem. Such an ecosystem, for example, may be in a host living organism, or a particular region within a host living organism.

The term “related chemical species,” as used herein, generally refers to any chemical species by which a grouping of related microbes may be identified. In some cases, a related chemical species may indicate the composition (e.g., abundance of microbes, type of microbes, etc.) of a group of related microbes within a microbial community. In other cases or in parallel, a related chemical species may indicate the functionality of a grouping of related microbes within a microbial community.

The terms “taxa,” or “taxon,” as used herein, generally refers to a group of similar microbes. Microbes may be classified into taxa by a host of different types of similarities. Several exemplary classification schemes are described below.

The term “unadministered subject(s),” as used herein, generally refers to a subject in which a test agent has not been administered. Such subjects may receive, instead, control agents or vehicles. In some cases, an unadministered subject has been administered no type of agent.

Host organisms are often exposed to exogenous agents (e.g., drugs, nutritional supplements, foods, sources of water, cosmetics, hygiene products, wound dressings such as bandages, topical antiseptics such as hydrogen peroxide, or topical pain relievers such as Epsom salts) without consideration to how such agents may affect various microbial communities that reside within the host. Moreover, the impact of an agent on the composition and/or functionality of a subject's microbiota may not be thoroughly considered during the development of an agent, such as in the case of the development of a therapeutic drug, such as an antibiotic. Indeed, many complications with the consumption of or exposure to a particular agent may be due to unfavorable disruption of microbiota.

Shortcomings to assessing the impact of an agent on microbiota may be due to the lack of available, reproducible, and standardized methods for assessing the differential impact of an agent, on the composition and functionality of various microbial communities within a living organism. In one instance, the successful development of such methods requires that various challenges be overcome including the fact that many microbial communities are often characterized by intrinsic variations, both across host species and across time. In another instance, safety regulations regarding agent use in humans may make it difficult to assess the differential impact of unapproved agents. Nevertheless, the successful development of reliable methods that enable accurate, reproducible assessment of the disruptive potential of agents on microbial communities of a living organism could offer an important tool that could be used to assess the utility of already available agents, agents currently in development, and agents to be developed in the future.

Recognized herein is a need for methods for reproducible assessment of the differential impact of agents on the composition and functionality of various microbial communities that reside within a host living organism. Composition of a microbial community may generally refer to the makeup of a microbial community and may include either or both of the number of microbes and types of microbes of the particular microbial community. Functionality of a microbial community may generally refer to the capability of a microbial community to exercise regular activities with non-limiting examples that include metabolism, respiration, and gene expression.

This disclosure provides methods and systems for characterizing the effects of one or more agents on at least one microbial organism of a living organism host. In one aspect, the disclosure provides methods for determining a quantitative measure of such effects, referred to herein as a Microbiome Modulation Index (MMI). Calculation of an MMI may rely on the enumeration of microbial taxa comprised in microbial communities found in subjects, when the subjects are exposed to one or more agents that may affect the composition of these microbial communities. Moreover, the calculation of an MMI may rely, for example, on the enumeration of a related chemical species associated with taxa of interest, such as, for example, gene expression or metabolic products. Enumerating related chemical species may be useful in assessing changes to either or both of abundance and functionality with respect to a given microbial taxon. Enumerations may be completed prior to, during, and after administration of an agent of interest.

In another aspect, the disclosure provides methods for generating (e.g., estimating) an MMI value in various species. Such methods can include the administration of an agent to a plurality of subjects of a first species in order to provide MMI values for subjects of the first species or for subjects of one or more other species. In cases where agents are administered to subjects of a first species, additional terms may be added to calculations in order to estimate an MMI value for subjects of a different species. In vitro methods may also be used to estimate an MMI for subjects of a given species, by applying an agent to in vitro cultures of relevant microbiota, and using MMIs generated in vitro as estimates for subjects of the given species.

In yet another aspect, the disclosure provides methods for both interpreting an MMI and enabling practical use of an MMI in a variety of applications.

### Microbiome Modulation Index (MMI)

This disclosure provides methods for generating a Microbiome Modulation Index (MMI) for an agent. Generally, an MMI may be calculated for an agent by enumerating the abundance of one or more microbial taxa and/or enumerating the functionality of one or more microbial taxa in samples obtained from at least one subject that has been administered an agent. Microbial taxa, for example, may be enumerated via a related chemical species (e.g., products of microbial metabolism, respiration, or gene expression) associated with the microbial taxa. Detection (e.g., quantitative detection) of a related chemical species associated with microbial taxa may quantitatively indicate the presence (e.g., abundance) of a given microbial taxon and/or quantitatively indicate the functionality of the given microbial taxon. Enumerations are generally completed for samples obtained both prior to and at some point after administration of the agent to the subject. These enumerations are then entered into one or more algorithms that may be used to generate an MMI for the agent. In general, enumerations in unadministered subjects (e.g., subjects administered control agents such as phosphate buffered saline, a placebo, or subjects administered no agent, etc.) may be included in algorithms used to generated an MMI.

In addition to including enumerations described above, algorithms used to generate an MMI may also address the variability of each particular microbial taxon and/or related chemical species that is observed in a plurality of unadministered subjects (e.g., subjects not exposed to an agent). In some examples, this may be accomplished by adding a variability weight, gᵢ, to algorithms used to calculate the MMI. A variability weight may be added for one or more enumerated microbial taxon and/or related chemical species. Variability may, for example, be expressed as the standard deviation, standard error, or variance of enumerations obtained at specific time points during monitoring. Moreover, an average variability may be calculated from individual variabilities measured in each test subject.

In some cases, a variability weight and can be obtained by monitoring the variability in the relative abundance of the appropriate microbial taxon in samples obtained from unadministered subjects across a similar period of time, or a relatively different period of time, used to monitor samples from test subjects administered with the agent of interest. Relative abundance (RA) of a microbial taxon, may be calculated, for example, according to exemplary Equation 1:

$$RA = \frac{{N}_{taxon}}{{N}_{total}} \qquad ( 1 )$$

where Nₜₐₓₒₙ is the number of microbes for the taxon of interest observed; and

where Nₜₒₜₐₗ is the number of total microbes observed.

An analogous calculation can be made for calculating the relative abundance of enumerated related chemical species.

The number of time points used to generate a variability weight may vary. For example, the relative abundance of a microbial taxon or related chemical species may be determined from samples obtained from unadministered subjects at the same time point used to obtain pre-administration samples from administered subjects and again at the same time point used to obtain post-administration samples from the administered subjects. The difference in relative abundance between the two time points may be considered the observed variability. Alternatively, samples may be taken for a greater number of time points. In some examples, the number of time points used to generate a variability weight is from about 1 time point to about 30 time points. In other examples the number of time points used to generate a variability weight is from about 1 time point to about 20 time points. In other examples, the number of time points used to generate a variability weight is from about 1 time point to about 10 time points. In still other examples, the number of time points used to generate a variability weight is about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 time points.

In some examples, the number of time points used to generate a variability weight is at least 30 time points. In other examples the number of time points used to generate a variability weight is at least 20 time points. In other examples, the number of time points used to generate a variability weight is at least 10 time points. In other examples, the number of time points used to generate a variability weight is at least 5 time points. In still other examples, the number of time points used to generate a variability weight is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 time points.

The time interval between time points used to generate a variability weight may vary. Intervals may be equally spaced between time points (e.g., for example, a time point is taken every 5 minutes) or intervals may be spaced such that intervals are different between different time points. Moreover, the duration of a time interval may vary. In some examples, the duration of the time interval may be from about 1 min to about 5 days. In other examples, the duration of the time interval may be from about 6 hours to about 5 days. In other examples, the duration of the time interval may be from about 12 hours to about 5 days. In other examples, the duration of the time interval may be from about 1 day to about 5 days. In still other examples, the duration of the time interval may be about 0.1, 0.2, 0.3, 0.4, 0.5, 0.6, 0.7, 0.8, 0.9, 1, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 6.0, 7.0, 8.0, 9.0, 10, 12, 14, 16, 18, or 20 days.

In some examples, the duration of the time interval may be at least 5 days. In other examples, the duration of the time interval may be at least 3 days. In other examples, the duration of the time interval may be at least 1 day. In other examples, the duration of the time interval may be at least 0.1 days. In still other examples, the duration of the time interval may be at least 0.1, 0.2, 0.3, 0.4, 0.5, 0.6, 0.7, 0.8, 0.9, 1, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 6.0, 7.0, 8.0, 9.0, 10, 12, 14, 16, 18, or 20 days.

The number of subjects used to calculate a variability weight may vary. In some examples, the number of subjects used to calculate a variability weight is from about 3 subjects to about 100 subjects. In other examples, the number of subjects used to calculate a variability weight is from about 3 subjects to about 30 subjects. In other examples, the number of subjects used to calculate a variability weight is from about 3 subjects to about 10 subjects. In still other examples, the number of subjects used to calculate a variability weight is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 subjects.

In some examples, the number of subjects used to calculate a variability weight is at least 100 subjects. In other examples, the number of subjects used to calculate a variability weight is at least 30 subjects. In other examples, the number of subjects used to calculate a variability weight is at least 5 subjects. In still other examples, the number of subjects used to calculate a variability weight is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 subjects.

In some examples, the number of samples used to calculate a variability weight is at least 100 samples. In other examples, the number of samples used to calculate a variability weight is at least 30 samples. In other examples, the number of samples used to calculate a variability weight is at least 5 samples. In still other examples, the number of samples used to calculate a variability weight is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 samples.

A variability weight (g) may be calculated for a microbial taxon or related chemical species, for example, in samples obtained from a plurality of unadministered subjects according to exemplary Equation 2:

gᵢ=1−δ/δₘₐₓ (2)
where δ is the variance and is calculated for each microbial taxon or related chemical species according to exemplary Equation 3:

$$δ = \frac{{Σ}_{i}^{n} \vert {R}_{1 , i} - {R}_{0 , i} \vert}{n} \qquad ( 3 )$$

where R_{1,i} is the relative abundance of the appropriate microbial taxa or related chemical species in a sample obtained from unadministered subject i at time-point 1, corresponding to the same time point used to obtain post-administration samples from administered test subjects,
where R_{0,i} is the relative abundance of the appropriate microbial taxa or related chemical species in a sample obtained from unadministered subject i at time-point 0, corresponding to the same time point used to obtain samples from pre-administration samples from administered test subjects,
where n is the total number of unadministered subjects observed for a given microbial taxon or related chemical species.
wherein δₘₐₓ is the value the largest value of δ obtained for all microbial taxa or related chemical species enumerated. In accordance with the above Equation 2, gᵢ=0 for the most variable microbial taxa or chemical species over time and gᵢ=1 for microbial taxa or related chemical species that do not change with time.

Estimates of MMI values for an agent in unadministered subjects of one or more different species from a test species may be obtained from administration of an agent to subjects of the test species. For example, estimates of an MMI for an agent administered to humans (e.g., the unadministered subjects of a different species from the test species) may be obtained by administering the agent to mice (e.g., subjects of the test species) and calculating an MMI for the agent with respect to mice. The MMI generated for the agent in mice can be used to estimate the MMI for the agent in humans. Subjects of the test species may be wild-type, genetically-modified, or gnotobiotic. An example of a gnotobiotic test species, may be, for example, a murine species free of naturally occurring murine microbiota and subsequently transplanted with human microbiota such that its microbiome is humanized.

Estimates of MMI values for an agent in unadministered subjects may be obtained from exposing an agent to in vitro cultures of appropriate microbiota. For example, estimates of an MMI for an agent administered to humans may be obtained by applying the agent to test cultures of appropriate human microbiota. The MMI generated for the in vitro culture can be used to estimate the MMI for the agent in humans.

MMI values determined in subjects of a test species or in vitro culture may be directly extrapolated to unadministered subjects of a different species from the test species. In other words, the generated MMI for the agent in subjects of the test species may be considered the estimated MMI value for the agent in non-administered subjects of a different species. Alternatively, algorithms used to calculate an MMI may include terms that characterize the non-administered species. Such algorithms can be used to generate an MMI for an agent in unadministered subjects of a different species than the test species, using enumerations of samples obtained from subjects of the test species. For example, an algorithm may comprise a prevalence weight, fᵢ that describes the relative abundance of a microbial taxon or related chemical species in one or more unadministered subjects of the non-test species. A prevalence weight may be calculated, for example, from samples obtained from a plurality of unadministered subjects of the non-test species. In some examples, the prevalence weight for a microbial taxon or related chemical species may be calculated according to exemplary Equation 4:

$${f}_{i} = \frac{{A}_{x}}{{A}_{n}} \qquad ( 4 )$$

wherein Aₓ is the number of samples in which the abundance of the microbial taxon or related chemical species is at or above a threshold value x.
wherein Aₙ is the total number of samples in which the microbial taxa or related chemical species was enumerated.
The threshold value x is determined as the value at which some level of confidence can be obtained that the microbial taxon or related chemical species would be found in a subsequent sample from the same source. In accordance with Equation 3, fᵢ=1 implies that a microbial taxon is sufficiently abundant in all samples and fᵢ=0 implies that a microbial taxon was sufficiently abundant in none of the samples.

The number of subjects used to calculate a prevalence weight may vary. In some examples, the number of subjects used to calculate a prevalence weight is from 3 subjects to about 100 subjects. In other examples, the number of subjects used to calculate a prevalence weight is from about 3 subjects to about 30 subjects. In other examples, the number of subjects used to calculate a prevalence weight is from about 3 subjects to about 10 subjects. In still other examples, the number of subjects used to calculate a prevalence weight is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 subjects.

The number of subjects used to calculate a prevalence weight may vary. In some examples, the number of subjects used to calculate a prevalence weight is at least 100 subjects. In other examples, the number of subjects used to calculate a prevalence weight is at least 30 subjects. In other examples, the number of subjects used to calculate a prevalence weight is at least 5 subjects. In still other examples, the number of subjects used to calculate a prevalence weight is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 subjects.

The number of samples used to calculate a prevalence weight may vary. In some examples, the number of samples used to calculate a prevalence weight is at least 100 samples. In other examples, the number of samples used to calculate a prevalence weight is at least 30 samples. In other examples, the number of samples used to calculate a prevalence weight is at least 5 samples. In still other examples, the number of samples used to calculate a prevalence weight is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 samples.

Estimates of MMI values specific for a condition of interest (e.g., obesity, type 2 diabetes, glucose intolerance) may be obtained as well. Those microbial taxa or related chemical species known to be associated with the condition, or found to differ in prevalence corresponding to severity or extent of the condition, can be highlighted in MMI calculations specific for that condition. For example, an algorithm may comprise a condition importance weight, hᵢ, that describes the importance of a microbial taxon or related chemical species in a condition of interest. A condition importance weight for a microbial taxon or related chemical species may be calculated, for example, from prior research on a condition of interest. In one example, a plurality of samples may be obtained from subjects having a condition of interest without administration of an agent to the subjects. The relevant microbial taxon may be surveyed in each of the samples. In cases where the relevant microbial taxon is present at or exceeding a threshold level, the microbial taxon is considered associated with the condition. For example, a threshold level may be determined by measuring the relevant taxon abundance in samples obtained from subjects lacking the condition of interest. The relevant taxon abundance determined from samples obtained from subjects lacking the condition of interest could be used as a threshold level. For example, where taxon abundance were to fall outside of the abundance observed in subjects lacking a condition of interest, the taxon could be considered as related to the condition of interest. A condition importance weight can then be calculated according to exemplary Equation 5:

$${h}_{i} = \frac{c}{N} \qquad ( 5 )$$

wherein c is the number of instances where the microbial taxon or related chemical species was associated with the condition of interest, and
wherein N is the total number of samples surveyed.

In some cases, the number of instances where the microbial taxon or related chemical species was associated with the condition of interest can be determined, for example, via a threshold level as described above. For example, if the threshold level associated with a condition of interest is 10%±2% of a microbial taxon, then the number of samples with >12% or <8% of the microbial taxon may be a measure of the number of instances where the microbial taxon or related chemical species was associated with the condition of interest (e.g., c as described above in Equation 5).

An algorithm to calculate an MMI for an agent may incorporate any of a variability weight, a prevalence weight, and/or a condition importance weight in order to account for the variability of analyzed microbial taxa or related chemical species in administered subjects of a test species, the importance of the particular microbial taxa or related chemical species in unadministered subjects of a non-test species, and the importance of particular microbial taxa or related chemical species in a condition of interest, respectively. For example, an agent may be administered to mice for calculation of the MMI, and, thus, a variability weight calculated for a plurality of unadministered mice. Moreover, enumerations of microbial taxa in mice may be used to estimate an MMI for the agent in a human. Thus, prevalence weights, for enumerated microbial taxa may be included in an algorithm used to estimate the MMI for the agent in humans. Furthermore, the MMI for the agent may also be used to estimate the MMI in humans with a particular condition. Thus, condition importance weight may be employed. Or in another example, all three weights may be used to generate an MMI for humans in mice, using the variability, prevalence, and condition weights described above.

An exemplary algorithm used to estimate the MMI for an agent in a non-administered, first species with a condition by administering the agent to subjects of a second species is shown collectively in Equations 6 and 7. First, an intermediate value d is calculated for each test subject administered with the agent. The abundance of each microbial taxa analyzed is enumerated before and after administration of the agent and the value d is calculated according to exemplary Equation 6:

$$d = \frac{{Σ}_{i} {f}_{i} * {g}_{i} * {h}_{i} * \vert {A}_{1 i} - {A}_{0 i} \vert}{{Σ}_{i} ( {A}_{1 i} + {A}_{0 i} )} \qquad ( 6 )$$

wherein hᵢ is a condition importance weight of microbial taxon or related chemical species i,
wherein gᵢ is a variability weight of microbial taxon or related chemical species i in unadministered subjects of the second species,
wherein fᵢ is a prevalence weight of microbial taxon or related chemical species i in unadministered subjects of the second species,
wherein A₁ᵢ is the abundance of microbial taxa or related chemical species i in a sample obtained from the subject at time-point 1 after administration of the agent to the subject, and
wherein A₀ᵢ is the abundance of microbial taxa or related chemical species i in a sample obtained from the subject at time-point 0, prior to the administration of the agent to the subject.

Following the calculation of d for each test subject administered the agent, the result of Equation 6 is then used to calculate an MMI according to exemplary Equation 7:

MMI= d̅/d₀ (7)

wherein d̅ is the average d calculated from all subjects administered the test agent, and

wherein d₀ is the average d calculated from a plurality of unadministered control subjects (e.g., subjects not administered the test agent) kept at the same conditions as those administered with the agent.

In some examples, the UniFrac metric may be incorporated into algorithms used to generate an MMI. UniFrac metrics generally refers to a metric that is a distance measure between organismal communities using phylogenetic information.

The variability weight, gᵢ, prevalence weight, fᵢ, and condition importance weight hᵢ may each be optionally omitted from Equation 6 in calculating d for each subject administered a test agent. In the case of omitting fᵢ, calculations for d would be used to calculate MMI values for subjects of the test species, without considering the relevance of each microbial taxon or related chemical species in the first, non-administered species. In the case of omitting gᵢ, MMI values obtained from Equation 6 would not address the variability in relative abundance of the given microbial taxon or related chemical species in the test species. In the case of omitting hᵢ, MMI values obtained from Equation 6 would not address the condition importance of the given microbial taxon or related chemical species.

The number of test subjects administered with a test agent to determine an MMI may vary. In some examples, the number of test subjects administered with a test agent to determine an MMI for that agent may be from about 1 subject to about 100 subjects. In other examples, the number of test subjects administered with a test agent to determine an MMI for that agent may be from about 1 subject to about 50 subjects. In other examples, the number of test subjects administered with a test agent may be from about 1 subject to about 10 subjects. In still other examples, the number of test subjects administered with a test agent may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 test subjects.

In some examples, the number of test subjects administered with a test agent to determine an MMI for that agent may be at least 100 subjects. In other examples, the number of test subjects administered with a test agent to determine an MMI for that agent may be at least 50 subjects. In other examples, the number of test subjects administered with a test agent may be at least 5 subjects. In still other examples, the number of test subjects administered with a test agent may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 test subjects.

In some examples, the number of samples analyzed to determine an MMI for an agent may be at least 100 samples. In other examples, the number of samples analyzed to determine an MMI for an agent may be at least 50 samples. In other examples, the number of samples analyzed to determine an MMI for an agent may be at least 5 samples. In still other examples, the number of samples analyzed to determine an MMI for an agent may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 test subjects.

An MMI may be determined for an agent by administering the agent to subjects of various living organisms. Any living organism capable of being administered a given agent may be used. Non-limiting examples of such living organisms include: species of a mammal, species of a rodent, species of a mouse, species of a rat, species of a dog, species of a cat, species of a hamster, species of a monkey, species of a pig, species of a squirrel, species a guinea pig, species of a gerbil, species of a bird, species of hydra, species of rabbit, species of fish, species of frog, species of cow, species of lobster, species of lamb, species of chicken, species of Drosphilia, species of Xenopus, livestock, a companion animal, and a human. In some examples, the living organism used to generate an MMI is a species of a common laboratory animal, such as a species of mouse or rat. Moreover, a living organism may be a wild-type species or may be a genetically-modified species. Species may also be gnotobiotic. A gnotobiotic species may be, for example, a murine species lacking microbiota that is transplanted with human microbiota.

An MMI value determined for an agent by administering the agent to subjects of one species of a living organism may be used to generate (e.g., estimate) the MMI for the agent in subjects of another species of a living organism. Any living organism in which enumerated taxa or related chemical species are present may have an estimated MMI generated. Non-limiting examples of species in which MMI values may be estimated by determining an MMI in an alternative species include: species of a mammal, species of a rodent, species of a mouse, species of a rat, species of a dog, species of a cat, species of a hamster, species of a monkey, species of a pig, species of a squirrel, species a guinea pig, species of a gerbil, species of a bird, species of hydra, species of rabbit, species of fish, species of frog, species of cow, species of lobster, species of lamb, species of chicken, species of Drosphilia, species of Xenopus, livestock, a companion animal, and a human.

The pairings of species, such that an MMI calculated for a subject of one species is used to generate the MMI for a subject of a different species, may vary. In some examples, the two species may be species of differing types of organisms. For example, an MMI estimate may be generated for a human by determining an MMI in a species of a common laboratory animal such as a mouse or rat. Non-limiting examples of species pairs used to generate an MMI include: a human and a species of mouse; a human and a species of rat; a human and a species of dog; a human and a species of monkey; a human and a species of rabbit; a human and a species of pig; a dog and a species of mouse; a dog and a species of rat; a cat and a species of mouse; a cat and a species of rat; and so on. Any two organisms (including those selected from the exemplary organisms described herein) may be paired.

In other examples, an MMI estimate for an agent in a different species of the same type of living organism tested may be obtained. For example, the MMI obtained for an agent in one species of a mouse may be used to generate an estimate for an MMI value for the agent in another species of mouse.

An MMI value determined for an agent by administering the agent to in vitro systems may be used to estimate the MMI for the agent in a living organism. Non-limiting examples of such systems include co-cultures (e.g., intestinal epithelial cells co-cultured with bacteria), mixed microbial community culture systems (e.g., fecal fermentation), intestinal simulator systems with fecal fermentation. Any of a prevalence weight, variability weight, and condition importance weight may all be included in MMI calculations that are generated from in vitro systems.

The number of species in which an estimate MMI is generated from a determined MMI can vary. In some examples, the number of species in which an MMI estimate is generated is from about 1 species to about 100 species. In other examples, the number of species in which an MMI estimate is generated is from about 1 to about 50 species. In other examples, the number of species in which an MMI estimate is generated is from about 1 to about 20 species. In other examples, the number of species in which an MMI estimate is generated is from about 1 to about 5 species. In still other examples the number of species in which an MMI estimate is generated is about 1, 2, 3, 4, 5, 6, 7, 8, 9, 10, 11, 12, 13, 14, 15, 16, 17, 18, 19, 20, 30, 40, 50, 60, 70, 80, 90, or 100 species.

Samples may be obtained from a variety of sources, including both internal environments and body cavities. Non-limiting examples of sample sources include the gut, the vagina (including the cervix), the respiratory system, the ear, nasal passages, an oral cavity, a sinus, a nare, the urogenital tract, skin, feces, udders, auditory canal, earwax, breast milk, blood, sputum, urine, saliva, open wounds, secretions from open wounds, and a combination thereof. In some examples, MMI values are obtained from samples that indirectly represent microbial communities in other parts of a living organism from which they were obtained. For example, samples from feces may be used to calculate MMI values for an agent with respect to microbial communities of the gut. Moreover, surgical means may be used to access internal tissues, such, as, for example, the gut.

A single sample may be obtained per subject administered an agent at each time point or multiple samples may be obtained per subject per time point. In cases where multiple samples are obtained, the samples may be pooled such that appropriate enumeration is completed on a pooled sample. Alternatively, each obtained sample may be separately evaluated, such that the enumeration reported for a particular subject at a given timepoint is an average of each enumeration obtained from each sample.

An MMI value may be determined for virtually any agent that may be administered to a living organism and/or applied to the surface of a living organism. Non-limiting examples of such agents include: a microbe, a related chemical species to a microbe, a virus, a prebiotic, a probiotic, a synbiotic, a fecal transplant, a drug, an antibiotic, a food, a beverage, a nutraceutical, a supplement, a beauty care product (e.g., makeup, hairspray, lotion, cosmetics, sunscreen, fragrances), personal hygiene product (e.g., shampoo, soap, shower gel, conditioner, chemically treated wipes, hand sanitizer), an allergen, a household chemical (e.g., bleach, ammonia, caustic household cleaning mixtures, fertilizer, gardening chemicals, paint, paint thinner, Scotchguard), wound dressings (e.g., bandages, liquid bandages), wound antiseptics (e.g., hydrogen peroxide), an industrial chemical (e.g., solvents, caustics, acids), a hazardous chemical, water from a municipal water source, an environmental sample (e.g., soil samples, water samples from natural sources), aerosols that may be inhaled via the nose or throat, topical pain relievers (e.g., Epsom salts), materials used to make clothing, and combinations thereof. In some cases, an agent may be generally recognized as safe (GRAS).

In some examples, an agent is administered orally, such that it is ingested via the mouth, which includes methods such as oral gavage. Alternatively, an agent may be administered topically such that it is applied to one or more outer surfaces of a living organism. Topical administration may be desired such that surface microbial communities are evaluated or such that the agent is absorbed into the internal compartment of the living organism, where microbial communities are studied. For example, an agent, such as a bandage, may be applied topically to the skin of a human. In another example, Epsom salt, used to sooth sore muscles, may be applied topically to the skin of a human such that it is absorbed and transported into In other instances, an agent may be administered intravenously, intrathecally, intrarectally (e.g., in the case of a fecal transplant), intraperitoneally, intradermally, or by inhalation.

An agent for which an MMI is calculated may be a therapeutic drug. The drug may be a drug already available in the marketplace, a drug previously available from the marketplace but subsequently withdrawn, a drug in development, or a chemical entity not already indicated as a potential drug. Non-limiting examples of such drugs include Prozac, Precose, Ambien, Mesalamine, Nexium, Seroquel, Cymbalta, Crestor, Lipitor, Plavix, Actos, glucophage (e.g., Metformin), Belviq, Qsymia, estrogen, a synthroid, lisinopril, lotensin, azithromycin, amoxicillin, Pentasa, Ritalin, Viagra, Diflucan, Prilosec, ibuprofen, aspirin, Ensure, Slim Fast, PediaSure, Claritin, Benadryl, or caffeine. For additional drugs for which a MMI may be calculated, see listings of drugs in reference materials such as the U.S. Food and Drug Administration Orange Book or Merck Medical Index, which are both incorporated herein in entirety by reference.

An MMI may be generated for an agent when administered in combination with one or more other agents. Agents of interest may be administered separately or may be administered simultaneously. For example, an MMI may be calculated for a drug when administered in combination with one or more other drugs. The number of agents that are combined with an agent for which an MMI is generated can vary. In some examples, the number of other agents combined with an agent to generate an MMI is from about 1 other agent to about 20 other agents. In other examples, the number of other agents combined with an agent to generate an MMI is from about 1 other agent to about 10 other agents. In other examples, the number of other agents combined with an agent to generate an MMI is from about 1 other agent to about 5 other agents. In still other examples, the number of other agents combined with an agent to generate an MMI for that agent is about 1, 2, 3, 4, 5, 6, 7, 8, 9, 10, 11, 12, 13, 14, 15, 16, 17, 18, 19, 20, 30, 40, or 50 other agents.

An MMI value may be calculated for a set of agents based on the individual component agents of the particular set. Agents of the set may be administered separately or may be administered simultaneously. For example, various foods of a diet may be administered to a living organism, either individually or in combinations, to generate an MMI value for the diet as a whole. An MMI for a set of agents may be calculated by a number of means including, for example, as the total sum of the MMIs for each agent in the set or by determining the average MMI of the agents in the set. Weights might also be added to the calculation to for example, emphasize particular agents of a set in calculating the set's MMI.

The number of microbial taxa or related chemical species that are enumerated to generate an MMI may vary. Number variance may vary, for example, due to the number of species or related chemical species that are present in a microbial community of interest. Some microbial communities of interest may possess greater numbers of relevant taxa or related chemical species that are present. In some examples, the number of microbial taxa or related chemical species that are enumerated to generate an MMI is from about 1 microbial taxa or related chemical species to about 1,000,000 microbial taxa or related chemical species. In other examples, the number of microbial taxa or related chemical species that are enumerated to generate an MMI is from about 1 microbial taxa or related chemical species to about 100,000 microbial taxa or related chemical species. In other examples, the number of microbial taxa or related chemical species that are enumerated to generate an MMI is from about 1 microbial taxa or related chemical species to about 10,000 microbial taxa or related chemical species. In other examples, the number of microbial taxa or related chemical species that are enumerated to generate an MMI is from about 1 microbial taxa or related chemical species to about 100 microbial taxa or related chemical species. In other examples, a single microbial taxon is enumerated to generate an MMI. In still other examples, the number of microbial taxa or related chemical species that are enumerated to generate an MMI is about 1, 10, 100, 1000, 10,000, 100,000, or 1,000,000 microbial taxa or related chemical species. In some examples, the number of microbial taxa or related chemical species that are enumerated to generate an MMI is at least 1, 10, 100, 10,000, 100,000, or 1,000,000 microbial taxa. Moreover, MMI values may be calculated by enumerating all possible microbial taxa within a given taxonomic classification scheme or all known chemicals species related to microbial taxa of interest. Alternatively, MMI values may be calculated by enumerating one or more particular subsets of all possible microbial taxa within a given taxonomic classification scheme or all known chemical species related to microbial taxa of interest.

In some examples, MMIs that are calculated by enumerating all possible microbial taxa within a given taxonomic classification scheme or all known chemical species related to microbial taxa of interest may give information (e.g., how taxa are affected when in contact with the agent) about all possible microbial taxa in subjects of the species in which the taxa or related chemical species are enumerated or in living organisms for which an estimated MMI is derived from MMIs generated. In other examples, MMIs that are calculated by enumerating a subset of all possible microbial taxa within a given taxonomic classification scheme or all possible related chemical species may give information about that subset in subjects of the species in which the microbial taxa or related chemical species are enumerated or in living organisms for which an estimated MMI is derived from MMIs generated. In still other examples, MMIs that are calculated by enumerating a single microbial taxon within a given taxonomic classification scheme or related chemical species may give information about that microbial taxon or related chemical species in subjects of the species in which the taxon or related chemical is enumerated or in living organisms for which an estimated MMI is derived from MMIs generated.

In other examples, an MMI may be derived from taxa determined from other experimental conditions. Such experiments may include experiments that have determined certain taxa that are important to a specific disease condition or health of a living organism in general.

### Microbial Taxa Classification Schemes

Microbial taxa may be classified according to a variety of different schemes and any classification scheme may be used to generate an MMI. Different classification schemes may result in taxa of different microbial compositions. Moreover, a particular taxon may comprise varied numbers of microbial species. In some examples, a microbial taxon may comprise a single microbial species. In other examples, a taxon may comprise from about 1 microbial species to about 1,000,000 microbial species. In other examples, a taxon may comprise from about 1 microbial species to about 10,000 microbial species. In other examples, a taxon may comprise from about 1 microbial species to about 100 microbial species. In other examples, a taxon may comprise from about 1 microbial species to about 10 microbial species. In still other examples, a taxon may comprise about 1, 10, 100, 1,000, 10,000, 100,000, or 1,000,000 microbial species. In still other examples, a taxon may comprise at least 1, 10, 100, 1,000, 10,000, 100,000, or 1,000,000 microbial species. Moreover, enumerated microbial taxa used to generate an MMI may vary in the number of component microbial species comprised in each microbial taxon.

Microbial taxa may be arranged according to parsimonious trees such that nodes of the trees are species ordered in an evolutionary hierarchy. Taxa may be grouped, for example, in clades according to descendants of a node in the tree, such that all descendants from a common ancestor (or node) are grouped within a microbial taxon. Sub-taxa may also be derived for nodes at lower levels of the tree in an analogous fashion. Alternatively, more complicated schemes may be used to distinguish taxa within a parsimonious tree.

Microbial taxa may be arranged according to classical Linnaean taxonomy. Linnaean taxonomy generally relies on ordering species at various ranks such that organisms at a given rank all share one or more common characteristic. A common characteristic, for example, may be a common anatomical or structural feature shared by members of a given taxon. Non-limiting examples of classical Linnaean taxonomy, in order of highest rank to lowest rank, include: domains, kingdoms, phylums, classes, orders, families, genera, or single species. In general, a genus name and species name indicates a unique species using classical Linnaean taxonomy.

Microbial taxa may be arranged as operational taxonomic units (OTUs). For a thorough description of arrangement of microbial taxa into OTUs, see U.S. Patent Application Publication No. 2012/0165215 and U.S. Patent Application Publication No. 2009/0291858 which are both incorporated in their entirety herein by reference. An operational taxon unit (OTU) refers to a group of one or more organisms that can be represented as a node in a clustering tree. The level of a cluster is determined by its hierarchical order. In some examples, an OTU is a group tentatively assumed to be a valid taxon for purposes of phylogenetic analysis. In other examples, an OTU is any of the extant taxonomic units under study. In other examples, an OTU is given a name and a rank. For example, an OTU can represent a domain, a sub-domain, a kingdom, a sub-kingdom, a phylum, a sub-phylum, a class, a sub-class, an order, a sub-order, a family, a subfamily, a genus, a subgenus, or a species. In some cases, OTUs can represent one or more organisms from the kingdoms eubacteria, protista, or fungi at any level of a hierarchal order. In other cases, an OTU represents a prokaryotic or fungal order. Moreover, OTUs may be derived for example by a common physical attribute shared by its component organisms or may be derived from evolutionary hierarchy.

Alternatively, OTUs may be derived by other means such as by clustering organisms into OTUs by identifying of one or more conserved genes and/or polynucleotide sequence homologies for shared genes comprised in a plurality of organisms to-be-clustered. Highly conserved polynucleotides usually show at least 80%, 85%, 90%, 92%, 94%, 95%, or 97% homology across a domain, kingdom, phylum, class, order, family or genus, respectively. The sequences of these polynucleotides can be used for determining evolutionary lineage or making a phylogenetic determination and are also known as phylogenetic markers.

A database of nucleic acid sequences may be used to organize organisms into particular OTUs based on one or more conserved genes and/or highly homologous nucleic acid sequences shared by a group of organisms. The choice of database that is used to assign organisms to OTUs is dependent on a number of factors with non-limiting examples that include the total number of sequences within the database, the length of the overall sequences or the length of highly conserved regions within the sequences listed in the database, and the quality of the sequences therein. Typically, databases with longer target regions of conserved sequence may generally contain a larger total number of possible sequences that can be compared. In some examples, the sequences in a database are at least 10, 50, 100, 200, 300, 400, 500, 600, 700, 800, 900, 1,000, 1,200, 1,400, 1,600, 1,800, 2,000, 4,000, 8,000, 16,000 or 24,000 nucleotides long. Moreover, databases with a larger number of sequences may generally provide greater numbers of sequences from which to choose. In some examples, a database contains at least 10,000, 20,000, 30,000, 40,000, 50,000, 60,000, 70,000, 80,000, 100,000, 200,000, 500,000, 1,000,000 or 2,000,000 sequences.

A database used for the selection of OTUs may comprise at least 50%, 60%, 70%, 80%, 90%, 95%, 96%, 97%, 98%, 99% or up to 100% of the known sequences of the organisms to be clustered into OTUs. The sequences for each individual organism in the database can include more than about 20%, 30%, 40%, 50%, 60%, 70%, 80%, 90%, 95%, or 100% of the genome of the organism, or of the non-redundant regions thereof.

A variety of existing databases may be used to assign organisms to an OTU based on nucleic acid sequences. A listing of almost 40,000 aligned 16S rRNA sequences greater than 1250 nucleotides in length can be found on the Greengenes web application (greengenes.secondgenome.com), a publicly accessible database run by the Greengenes Consortium. Other publicly accessible databases include GenBank, Michigan State University's ribosomal database project, the Max Planck Institute for Marine Microbiology's Silva database, and the National Institute of Health's NCBI. Proprietary sequence databases or combinations created by amalgamating the contents of two or more private and/or public databases can also be used to assign organisms to a given OTU.

As noted above, OTUs may be arranged by sequence homology of a conserved polynucleotide. The conserved polynucleotide may be from a highly conserved gene or the conserved polynucleotide may be from a highly conserved region of a gene with moderate or large sequence variation. Moreover, the highly conserved polynucleotide may be an intron, exon, or a linking section of nucleic acid that separates two genes.

The highly conserved polynucleotide used to assign organisms to OTUs may be a phylogenetic gene. Non-limiting examples of a phylogenetic gene includes the 5.8S ribosomal ribonucleic acid (rRNA) gene, 12S rRNA gene, 16S rRNA gene-prokaryotic, 16S rRNA gene-mitochondrial, 18S rRNA gene, 23S rRNA gene, 28S rRNA gene, gyrB gene, rpoB gene, fusA gene, recA gene, cox1 gene, and the nifD gene. For eukaryotic species, rRNA genes can be nuclear, mitochondrial, or both. In some cases, the spacer region between highly conserved segments of two genes can be used. For example, the internal transcribed spacer (ITS) region between 16S and 23S rRNA genes can be used to differentiate closely related taxa with or without consideration of other rRNA genes, including conserved sections of either the 16S or 23S rRNA.

Due to structural constraints necessary for proper functioning of 16S rRNA when comprised in protein synthesis machinery (e.g., ribosomes), specific regions throughout the gene have a highly conserved polynucleotide sequence although non-structural segments may have a high degree of variability. Regions of the 16S rRNA gene that possess high levels of variability include the V1, V2, V3, V4, V5, V6, V7, V8, and V9 regions of the gene. These and other regions of high variability may be detected, for example, to distinguish/enumerate OTUs at a single species level, while regions of less variability might be used to distinguish OTUs that represent a subgenus, a genus, a subfamily, a family, a sub-order, an order, a sub-class, a class, a sub-phylum, a phylum, a sub-kingdom, or a kingdom. Such a classification scheme may be useful for identifying closely related microorganisms and OTUs from a background or pool of closely related organisms.

Microbial taxa may be arranged by virtue of other descriptors with non-limiting examples that include genomes, transcriptomes, proteomes, metabolomes, and metagenomes. Such descriptors may be both indicators of microbial compositions and functionality. In some examples, microbial organisms may be arranged into taxa via clusters of organisms with similar, full or partial transcriptomes. Transcriptomes generally refer to a set of ribonucleic acid (RNA) molecules of a living organism. RNA molecules may include messenger RNA (mRNA), ribosomal RNA (rRNA), transfer RNA (tRNA), and other non-coding RNA. Transcriptomes may be an entire set of all RNA molecules of a living organism or may be a particular subset of RNA molecules. Moreover, taxa may be arranged based on full organism transcriptomes or may be based on partial transcriptomes.

In some examples, microbial organisms may be arranged into taxa via clusters of organisms with similar proteomes. A proteome generally refers to a set of proteins expressed by a living organism. Proteomes may be an entire set of all proteins of a living organism or may be a particular subset of proteins. Moreover, taxa may be arranged based on full organism proteomes or may be based on partial proteomes.

In some examples, microbial organisms may be arranged into taxa via clusters of organisms with similar metabolomes. A metabolome generally refers to a set of small-molecule metabolites (such as metabolic intermediates, hormones and other signaling molecules, and secondary metabolites) found within a living organism. Metabolomes may be an entire set of all metabolites found within a living organism or may be a particular subset of metabolites. Moreover, taxa may be arranged based on full organism metabolomes or may be based on partial metabolomes.

In some examples, microbial organisms may be arranged into taxa via clusters of organisms with similar metagenomes. A metagenome generally refers to genetic material recovered directly from environmental samples, such as for example a living organism. Metagenomes may be an entire set of all genetic material found within a living organism or may be a particular subset of genetic material. Moreover, taxa may be arranged based on full metagenomes or may be arranged based on partial metagenomes.

### Enumerating Microbial Taxa and Related Chemical Species

Microbial taxa may be enumerated by a variety of means depending upon the desired route and/or available instrumentation. In some cases, microbial taxa may be enumerated by quantitatively detecting one or more related chemical species associated with a given microbial taxon in a sample. Moreover, enumerations of related chemical species may be used to indicate function of a given microbial taxon. Non-limiting examples of such chemical species include small-molecules (including metabolites and other species of molecular weight <1000 Da), peptides (e.g., up to 2, 3, 4, 5, 6, 7, 8, 9, 10, 11, 12, 13, 14, 15, 16, 17, 18, 19, 20, or more amino acids long), proteins, lipids, nucleic acids, and/or carbohydrates.

In some cases, a detected molecule may be a common structural component of a group of organisms comprised in a microbial taxon. For example, a protein type or lipid associated with the plasma membrane of a microbe may be detected. In addition, a molecule secreted, such as a metabolite, may be detected. For example, some bacteria are known to produce short-chain fatty acids such as butyrate, propionate, valerate, and acetate. Secretion of a species such as butyrate, for example, may be the common characteristic used to group organisms into a given microbial taxon. The detection of butyrate may then be used to enumerate the abundance of the respective microbial taxon in a sample. Moreover, a molecule, for example, may be a common metabolite produced by organisms within a given microbial taxon. Detection of that metabolite may then be used to enumerate the abundance of that microbial taxon in a sample and/or the functionality of that taxon. Furthermore, detection of one or more molecules in combination may be used to enumerate a microbial taxon.

Detection of a molecule, including a related chemical species, may be achieved with a variety of methods that include spectroscopic methods. Non-limiting examples of spectroscopic methods that may be used in enumerating microbial taxa include optical methods (e.g., UV-Vis absorbance, fluorescence, bioluminescence, Fourier-transform infrared (FT-IR) spectroscopy), nuclear magnetic resonance (NMR) spectroscopy, dynamic light scattering, and mass spectrometry.

Nucleic acids may be detected and quantified in order to enumerate microbial taxa. Such methods may be especially useful in cases where microbial taxa are OTUs distinguished by one or more gene sequence homologies. Detected nucleic acids may be deoxyribonucleic acid (DNA), ribonucleic acid (RNA), or combinations thereof. Nucleic acids may be detected generically, without respect to sequence, or may be detected in a sequence specific manner. In cases where sequence specific detection is desired, detection of a nucleic acid may be completed by the detection of a full-length gene sequence or may be completed by the detection of a partial-length gene sequence.

Moreover, nucleic acids may be downstream molecules synthesized as the result of gene transcription and/or metagenomic molecules present in a living organism. In general, a metagenomic molecule may be a genetic molecule that may be recovered from an environmental sample, such as a living organism. For example, in the case of the 16S rRNA gene, genomic DNA corresponding, in whole or part, to regions of the 16S rRNA gene, messenger RNA (mRNA) transcripts, in whole or part, of the 16S rRNA gene, and/or functional 16S rRNA may be detected and used to enumerate the abundance of a microbial taxon characterized by sequence homology of a particular 16S rRNA gene sequence.

Nucleic acid sequencing methods may be used to detect and quantify sequence specific nucleic acids such that they are used to enumerate the abundance of a microbial taxon characterized by homology of the detected sequence amongst organisms clustered into the microbial taxon. Non-limiting examples of sequencing methods that may be used include shotgun sequencing, polymerase chain reaction, real-time polymerase chain reaction, ligase chain reaction, single-molecule real-time sequencing, ion torrent sequencing, pyrosequencing, sequencing by synthesis, sequencing by ligation, chain termination sequencing, massively parallel signature sequencing, polony sequencing, SOLiD sequencing, DNA nanoball sequencing, heliscope single molecule sequencing, single molecule real time sequencing, nanopore sequencing, mass spectrometry sequencing, microfluidic sequencing, high-throughput sequencing, Illumina sequencing, HiSeq sequencing, MiSeq sequencing, or combinations thereof. Sequencing may be completed such that full-length genes are sequenced or partial-length genes are sequenced.

Sequence-specific detection of nucleic acids may also be completed with oligonucleotide probes. An oligonucleotide probe may be capable of hybridizing with a full-length or partial-length gene sequence of interest. Moreover, an oligonucleotide probe may be labeled with a detectable tag, such as a fluorescent dye, that may be detected. Alternatively, nucleic acid to be probed may be labeled such that its binding with the oligonucleotide probe is detected (via an attached label). An oligonucleotide probe may be a primer or a longer, different type of oligonucleotide. The oligonucleotide probe may the same type of nucleic acid as the target (e.g., DNA target and DNA oligonucleotide) or the oligonucleotide probe may be a different type of nucleic acid than the target (e.g., DNA target and RNA probe). Non-limiting examples of a label linked to an oligonucleotide probe may be a fluorescent dye, absorbent chemical species, radiolabel, quantum dot, or nanoparticle. Moreover, an oligonucleotide probe may also include a quencher (a molecule used, for example, to inhibit fluorescence). Probes useful in real-time polymerase chain reactions may be useful in sequence specific detection. Non-limiting examples of such probes include TaqMan probes, TaqMan Tamara probes, TaqMan MGB probes, or Lion probes.

Oligonucleotide probes may be immobilized to an array such that the binding of a target nucleic acid sequence is detected. In some examples, such oligonucleotide probes may be immobilized in one or more arrays. Each oligonucleotide probe is assigned a specific position in the array such that the position corresponds to the oligonucleotide probe. Nucleic acids to-be-detected may be labeled with an agent capable of being detected. Hybridization of a labeled nucleic acids to a complementary, immobilized sequence results in accumulation of detectable label at the signal which can then be identified indirectly as presence of the a given sequence. Nucleic acids to-be-analyzed may be exposed to an oligonucleotide probe array without size reduction or may be fragmented in order to ensure that the size of the to-be-analyzed nucleic acid is more similar to the oligonucleotide probes arranged on the array. Size similarity may result in better nucleic acid binding to oligonucleotide probes of the array. Oligonucleotide probe arrays have been generated for taxonomic analyses based on the sequence-specific detection of nucleic acids. Non-limiting examples of such arrays include the G2 PhyloChip and G3 PhyloChip. The selection of oligonucleotide probes, the construction of each array, methods for obtaining data, and methods for analysis of data obtained from each array are described in detail in U.S. Patent Application Publication No. 2009/0291858 and U.S. Patent Application Publication No. 2012/0165215 which are both incorporated in entirety herein by reference.

Oligonucleotide probes may be immobilized on microbeads. Binding of nucleic acids to oligonucleotide probes arranged on microbeads and detection of such nucleic acids is completed in an analogous fashion to that mentioned above for oligonucleotides, such that nucleic acids to-be-analyzed are labeled and their hybridization with an oligonucleotide probe results in the accumulation of detectable signal that can be indirectly interpreted as the presence of a sequence specific region of nucleic acid. Again, nucleic acids to-be-analyzed may be exposed to oligonucleotide probes on microbeads without size reduction or may be fragmented in order to ensure that the size of the to-be-analyzed nucleic acid is more similar to the oligonucleotide probes arranged on the microbeads.

DNA barcording may aid in enumerating microbial taxa. DNA samples from multiple subjects and timepoints may be PCR amplified using primers that incorporate a unique DNA barcode in addition to the 16S rRNA priming sites. Produced amplicons may then be pooled together and sequenced in a single batch.

Enumeration of microbial taxa may also be achieved by other means such as analyzing proteomes, transcriptomes, metabolomes, or combinations thereof. For example, microbial RNA transcripts, proteins, non-16S genes, etc. may be profiled and their abundance used to determine the impact of the agent on the microbial communities. Any of analyzing proteomes, transcriptomes, metagenomes, or metabolomes may be used to generate an MMI based on either microbial taxa function or composition.

### Methods for Interpreting and Using a Microbiome Modulation Index

The disclosure provides methods for interpreting and/or utilizing an MMI generated for an agent. In general, MMI values may be considered in isolation (i.e., not with respect to any reference MMI) or may be made based by comparing a generated MMI values with one or more reference MMI values or other data regarding composition and/or functionality of the microbiome. Reference MMI values generally refer to MMI values used for comparison purposes with a generated MMI. For example, a reference MMI may be a previously generated MMI value for agents desired for comparison purposes, an MMI generated for an agent in a particular population, an MMI generated for an agent in the same class of agents as a test agent, an MMI generated for an agent in a subject (or group of subjects) at a previous time, an MMI for an agent generated in the absence of another agent. A reference MMI value may also be a threshold value generated, for example, empirically. In cases where threshold values are used for reference MMI values, threshold values may be considered with respect to the particular microbial taxa evaluated and/or any other available information (e.g., additional assays). In some examples, higher-than-threshold or reference MMI values may be desired. In other examples, lower-than-threshold or reference MMI values may be desired. Moreover, interpreting MMIs, either in isolation or by comparison to one or more other reference MMI values, may aid in decision-making regarding the utility of an agent in a variety of contexts that include, for example, health care and health safety.

When comparing two or more agents, at least one of the agents may be considered a reference agent for comparison purposes. The MMI value of the reference agent may be a reference MMI. In one example, the MMI for an existing agent (e.g., an existing drug) may be used as a reference MMI value for a new agent (e.g., a new drug). In other examples, a reference MMI may be an MMI determined for an agent when administered to a subject in a different amount, when administered to a subject using a different route of administration, or when administered to a subject in a different formulation.

MMIs used to make decisions may be generated in a species of interest, or, alternatively may be generated in a separate species such that MMIs used for decisions are estimates for a subject or subjects of a species of interest based upon the generated MMIs in the separate species. Moreover, decisions may be made for virtually any living organism for which MMIs or estimates of MMIs are available with non-limiting examples that include: a mammal, a species of a rodent, a species of a mouse, a species of a rat, a species of a dog, a species of a cat, a species of a hamster, a species of a monkey, a species of a pig, a species of a squirrel, a species a guinea pig, a species of a gerbil, a species of a bird, a species of hydra, a species of rabbit, a species of fish, a species of frog, a species of cow, a species of lobster, species of lamb, a species of chicken, a species of Drosphilia, a species of Xenopus, livestock, a companion animal, and a human. Altered states of existence, such as disease states, may also be considered such that MMI values are generated in subjects that can be characterized by the particular altered state of existence.

MMIs may aid in healthcare related decision-making with respect to the utility of an agent or a combination of agents. Decisions may include, for example, deciding upon the efficacy and/or safety of an agent or combination of agents. Non-limiting examples of such decisions include: determining the utility of a drug or combination of drugs; determining the utility of a supplement; determining the utility of a food or combination of foods (e.g., a diet); determining the utility of a beauty product or a combination of beauty products; determining the utility of a personal hygiene product or combination of personal hygiene products; determining the propensity of an agent to cause a condition associated with an undesirable shift in local microbial populations caused by the agent; and combinations thereof.

MMIs may aid in determining the utility of a drug or combination of drugs. Determining the utility of a drug or combination of drugs may include evaluating the efficacy and/or safety of a drug or combination of drugs. Such determinations may be made, for example, with respect to the capability of a drug or combination of drugs to cause a change in one or more microbial populations of a host administered the agent. In some cases, two drugs may be considered in combination therapy such that one drug aids in ameliorating the deleterious effects of the other. In some cases, it may be determined, based upon one or more MMI values for a drug or combination of drugs, that the drug or combination of drugs unfavorably alters one or more microbial populations of the host such that a drug or combination of drugs is determined to be no longer efficacious and/or unsafe. In cases where drugs are determined to be no longer efficacious and/or safe, it may be decided that treatment with the drug should cease. In other cases, it may be determined, based upon one or more MMI values, that the drug or combination of drugs favorably alters one or more microbial populations of the host such that the drug or combination of drugs is determined to be efficacious and/or safe. In cases where drugs are determined to be efficacious and/or safe, it may be decided that treatment with the drug should commence or continue. In still other cases, it may be determined that the drug or combination of drugs does not alter one or more microbial populations such that the efficacy and/or safety of a drug or combinations of drugs cannot be determined from MMIs. In any of these cases, a reference MMI may aid in decision-making.

For example, one or more MMIs may be generated for administering an experimental drug to a human using any of the methods described herein. The generated MMIs may then be compared with MMIs (generated in the same or similar fashion, with respect to substantially the same microbial taxa) generated for other approved drugs in the same class (e.g., chemical class, therapeutic class, etc.) as the experimental drug known to be safe and effective in humans. If similar MMI values are obtained for the experimental drug as are obtained for the approved drugs, then it may be decided that the experimental drug may also be safe and effective. If MMI values are obtained for the experimental drug that are substantially different than those for the known drugs, then, depending on the magnitude of the MMI and the particular microbial taxa evaluated, it may be determined that the experimental drug is not safe and/or effective or is safe and/or effective. Or, it may be determined that the experimental drug is more safe and/or more effective than the approved drugs. An analogous decision-making scheme could also be made for deciding upon the safety/efficacy of any combination of therapeutic agents. Moreover, a threshold MMI value with respect to efficacy/safety may be determined empirically and used for comparison with the MMI calculated for the experimental drug.

In another example, an MMI may be generated for an experimental drug administered to humans and the MMI indicative that the experimental drug is not safe in humans. The experimental drug may be then administered in combination with another agent and an MMI generated for the combination therapy in humans. The MMI for the combination therapy may be compared to the MMI for the experimental drug alone to decide whether or not the combination therapy helps to minimize the deleterious effects of the experimental drug when administered alone.

Determinations with respect to drugs can be made for a host of drug types. Drugs may include, for example, already approved drugs that are available on the market (prescription or over-the-counter), previously approved drugs that have been withdrawn for the market, drugs that are currently in pre-clinical (e.g., prior to a clinical trial) or clinical development, drugs that have yet-to-be-developed; and agents that are currently available but have not yet been considered for use as therapeutic drugs. Non-limiting examples of already available drugs that may be considered include: Prozac, Precose, Ambien, Mesalamine, Nexium, Seroquel, Cymbalta, Crestor, Lipitor, Plavix, Actos, glucophage (e.g., Metformin), Belviq, Qsymia, estrogen, a synthroid, lisinopril, lotensin, azithromycin, amoxicillin, Pentasa, Ritalin, Viagra, Diflucan, Prilosec, ibuprofen, aspirin, Ensure, Slim Fast, PediaSure, Claritin, Benadryl, or caffeine. For additional drugs for which a MMI may be calculated, see listings of drugs in reference materials such as the U.S. Food and Drug Administration Orange Book or Merck Medical Index, which are both incorporated herein in entirety by reference.

In particular, antibiotic therapies may be of especially important interest as they are generally designed to stunt growth the growth of or kill certain bacteria. Unfortunately, many antibiotics that treat harmful bacteria may also stunt the growth of or kill bacterial populations considered beneficial to the host. Thus, MMI values may be especially useful in determining the utility of an antibiotic or antibiotic used in combination with other drugs or agents.

Utility determinations may be made for supplements, beauty products, personal hygiene products, or any other type of agent in analogous fashion to that described above for drugs. Non-limiting examples of supplements include vitamins, nutraceuticals, prebiotics, probiotics, or synbiotics. Non-limiting examples of beauty products include makeup, hairspray, lotion, cosmetics, lip balm, sunscreen, and combinations thereof. Non-limiting examples of personal hygiene products include shampoo, soap, shower gel, conditioner, chemically treated wipes, and hand sanitizer. Determinations may also be made when any of these is considered with respect to a drug regimen. For example, MMI values may be used to determine the utility of one or more supplements and/or drugs when the supplement(s) and drug(s) are used in combination.

Determinations regarding the utility of drugs or supplements may be used to generate preferred regimens of treatments, that include the specific drug(s) utilized and/or appropriate doses (e.g., including dose levels or dosing frequency). For example, a drug regimen that includes a drug or combinations of drugs may be recommended, at particular doses, based upon determinations of MMIs. Recommendations may be based, for example, on ranked lists that may be generated based upon order of MMI values of a drug or a combination of MMI values for each drug in a combination therapy. Moreover, binary lists may also be generated such that lists of recommended and non-recommended drugs are compiled. Such lists may also include rankings within categories. Lists may be compiled such that recommended (or non-recommended) drug or combinations of drugs are those that have MMI values that are at or above (or below) a given threshold value or by comparison with one or more reference MMI values. In some cases, one or more MMI values may be used to adjust the dose of a drug or combination of drugs, such that acceptable dosing corresponds to acceptable MMI values. In some cases, a generated MMI may suggest that a drug should be dosed at lower than accepted levels to minimize safety issues with the drug.

Which side of a threshold that demarcates a recommended or non-recommended category may depend on the particular microbial communities that are altered when the host is exposed to a drug or combination of drugs of interest. For example, it may be that a drug or combination of drugs substantially alters the population of bacteria such that calculated MMI values are high. In some examples, high MMI values may be desired with respect to the microbial taxa evaluated and, thus, the drug or combination of drugs may be recommended. Alternatively, high MMI values may not be desired with respect to the microbial taxa evaluated and, thus, the drug or combination of drugs may not be recommended. In other cases, it may be that a drug or combination of drugs does not substantially alter the population of bacteria such that calculated MMI values are low. In such a case a drug or combination of drugs may also be recommended or not recommended, depending upon the particular microbial taxa evaluated.

Moreover, recommendations of agents may be formulated for particular populations of subjects. For example, the use of one or more drugs may be recommended (or not recommended) based on MMI values observed in subsets of populations. For example, it may be observed that particular microbiota respond differently to a drug in women than do the same microbiota in men when considering MMI values of the agent in the men and women populations. It may be the case that the drug is recommended only in women, for example, because the MMI is desirable only in women. In other words, subject selection for a particular agent or combination of agents may be achieved by using an MMI.

Determinations regarding the utility of a drug, a combination of drugs, a supplement, a combination of supplements, or a combination of supplements and drugs may be used during drug development or regulatory agencies (e.g., the U.S. Food and Drug Administration) to further evaluate the safety and/or efficacy of a drug. For example, during pre-clinical development (e.g., prior to a clinical trial) and clinical trials of a drug, MMIs may be used by a drug research and development organization to make decisions regarding initiating or continuing development of a particular compound into a therapeutic agent. For example, it may be determined that a particular class of agents may be useful as drugs to treat a given condition related to microbiota modulation, based on determined MMI values of the agents in a population of subjects. Novel drug leads may be generated from hits in the particular class for high-throughput screening and selection of clinical candidates.

Furthermore, MMI values may be used to select subjects for a clinical trial. For example, under the supervision of a drug research organization, an experimental drug may be given to a subject and an MMI favorable to the drug's intended action with respect to the evaluated microbial taxa is determined. Accordingly, the subject is then selected for a clinical trial for the experimental drug or selected to continue in a clinical trial for the experimental drug. In another example, an experimental drug may be given to a subject and an unfavorable MMI is generated such that it is determined that the drug is too toxic to the subject. Accordingly, the subject is removed from the clinical trial.

Moreover, at any step of the drug development process, MMIs might be used to assess the acceptability of an agent to receive approval for human use and/or the dosage at which an agent should be administered. Alternatively, MMI values might be used post-approval such that a regulatory agency makes a decision as to whether or not an approved drug should remain on the market or make changes to an already recommended dosage (including dosing level and frequency). For example, a drug known to have side-effects potentially linked to the abundance or functionality of one or more microbial taxa may have its MMI generated for humans. The MMI may be compared with MMIs for other drugs with similar mechanisms of action and/or directed to similar therapeutic targets in humans but known to have fewer or no side effects. In cases where the MMI for the agent associated with the side-effects is determined to be substantially different than those without the side-effects, it may be determined that the drug with the side-effects should be removed from approval.

MMIs may aid in determining a preference for a food (which may be a beverage) or a combination of foods, including a diet. Non-limiting examples of diets include the a South Beach Diet, a Dukin diet, a Stillman diet, an Atkins Diet, a gluten-free diet, a ketogenic diet, a low-residue diet, a liquid diet, a vegetarian diet, a low-calorie diet (e.g., Weight Watches, Jenny Craig, Nutrisystems), a low-fat diet, a low-carbohydrate diet, a low-protein diet, a low-monosodium glutamate (MSG) diet, a detox diet, an elimination diet, a specific carbohydrate diet, a diabetic diet, a dietary approaches to stop hypertension diet (DASH) diet, a best bet diet, an organic diet, and combinations thereof.

Determining a preference for a food or diet may include evaluating the safety of a food or diet and/or the propensity of a food or diet to cause a change in one or more microbial populations of a host administered the food or diet. In some cases, it may be determined, based upon one or more MMI values, that a food or diet unfavorably alters one or more microbial populations of the host such that a food or diet is determined to be not preferential and/or determined to be unsafe. In cases where a food or diet is determined to be not preferential and/or unsafe, it may be decided that ingesting the food or using the diet should cease. In other cases, it may be determined, based upon one or more MMI values, that a food or diet favorably alters one or more microbial populations of the host such that a food or diet is determined to be preferential and/or determined to be safe. In cases where a food or diet is determined to be preferential and safe, it may be decided that ingesting the food or using the diet should commence or continue. In still other cases, it may be determined that a food or diet does not alter one or more microbial populations such that the preference for and/or safety of a food or diet cannot be determined from MMIs

Determinations with respect to foods or diets can be made for virtually any type of food and or diet. In some cases, determinations are made between the same type of food that is obtained from a plurality of sources (e.g., beef obtained from grass-fed livestock vs. beef obtained from livestock fed on a concentrated diet of grain, soy, corn and other supplements such as steroids and antibiotics). Foods or diets may include, for example, already available foods or diets that are available on the market, foods or diets that have been withdrawn for the market, foods or diets that are currently in development, foods or diets that have yet-to-be-developed; and agents that are currently available but have not yet been considered for use as foods or as foods in a diet. Moreover, the combinations of a particular food or diet when administered in combination with a drug regimen and/or supplement regimen may also be evaluated by generating the appropriate MMIs values.

Determinations regarding the impact of a food or diet on one or more microbial communities in a host may be used to generate recommendations for foods or diets, based upon, for example, determinations of MMIs. Recommendations may be based, for example, on ranked lists of foods or diets that may be generated based upon order of MMI values of a food of MMI values for each food in a diet. Moreover, binary lists may also be generated such that lists of recommended and non-recommended drugs are compiled. Such lists may also include rankings within categories. Lists may be compiled such that recommended (or non-recommended) foods or diets are those that have MMI values that are at or above (or below) a given threshold value. Which side of a threshold that demarcates a recommended or non-recommended category may depend on the particular microbial communities that are altered when the host is exposed to a food or diet of interest. For example, it may be that a food or diet substantially alters the population of bacteria such that calculated MMI values are high. In such a case, a food or diet may be recommended. In other cases, it may be that a food or diet does not substantially alter the population of bacteria such that calculated MMI values are low. In such a case, a food or diet may also be recommended.

For example, a new combination of foods may be considered for a diet. An MMI may be generated for the combination of foods in humans. The determined MMI may be compared with respect to MMIs for diets known to be safe, yet alter substantially similar microbial taxa as those for which the new diet's MMI was determined. In cases where the MMI generated for the new diet is similar to the MMIs for diets known to function similarly and safely, a decision may be made that the diet would be preferential and/or safe. Alternatively, substantial deviations between the MMI of the new diet and those of the known, safe diets may indicate that either the new diet is unsafe or not-preferred or that the new diet is safer and possibly more preferred than those already known.

MMIs may aid in determining the propensity of an agent to cause one or more conditions. A number of conditions are known to be associated with the presence and composition of particular microbial communities. For example, the intestinal gut microbiota provides many crucial functions to its host, including contribution to digestion, the development of the immune system, and resistance to pathogenic colonization. Even a slight fluctuation in the symbiotic balance may be deleterious to the host, leading to pathological conditions such as, for example, Clostridium difficile infection or inflammatory bowel disease (IBD). As a result, it is important to monitor the effects of agents on microbiota as they may cause conditions to arise in an administered host. Other non-limiting examples of conditions that may be caused by an agent include a condition of the gut, Crohn's Disease (CD), irritable bowel syndrome (IBS), stomach ulcers, colitis, neonatal necrotizing enterocolitis, gastroesophageal reflux disease (GERD), gastroparesis, cystic fibrosis, chronic obstructive pulmonary disease, rhinitis, atopy, asthma, acne, a food allergy, obesity, periodontal disease, diarrhea, constipation, functional bloating, gastritis, lactose intolerance, visceral hyperalgesia, colic, pouchitis, diverticulitis, allergies, asthma, sinusitis, chronic obstructive pulmonary disorder (COPD), depression, attention deficit hyperactivity disorder (ADHD), autism, Alzheimers, migraines, multiple sclerosis (MS), Lupus, arthritis, Type 2 diabetes, obesity, non alcoholic steato hepatitis (NASH), non alcoholic fatty liver disease (NAFLD), risk of infarction/cardiovascular risk, heart failure, cancer, dental caries, gingivitis, oral cancer, oral mucositis, bacterial vaginosis, fertility, sinusitis, allergies, cystic fibrosis, lung cancer, psoriasis, atopic dermatitis, methicillin-resistant staphylococcus aureus (MRSA), or combinations thereof.

The capability of an agent to cause a condition can be evaluated with virtually any agent including drugs, supplements, nutritional supplements, beauty products, personal hygiene products, and foods described above. Moreover, agents may be household chemicals (e.g., bleach, ammonia, caustic household cleaning mixtures, fertilizer, gardening chemicals, paint, paint thinner, Scotchguard) or hazardous materials. Moreover, combinations of agents may also be evaluated. In cases where disease states are already present, the propensity of an agent to further exacerbate the symptoms of a condition may be evaluated. For example, it is generally known that the symptoms of irritable bowel syndrome (IBS) or Crohn's Disease (CD) are exacerbated with diets of complex carbohydrates. Thus, an MMI for a diet comprising a large fraction of complex carbohydrates may be used to determine that such a diet is not preferred.

For example, a new household chemical may be considered for consumer use. An MMI may be generated for the household chemical using samples (e.g., skin samples) obtained from humans. MMI values may be generated with calculations that include enumerations of microbial taxa known to be associated with atopic dermatitis and/or that may include condition importance weights for atopic dermatitis. The determined MMI may be compared with respect to a threshold MMI value at which modulation of the relevant microbial taxa is known to significantly increase the likelihood that a human subject would get atopic dermatitis. If the MMI generated for the household cleaner does not meet or exceed the threshold MMI, it may be determined that the household cleaner is not likely to cause atopic dermatitis. Alternatively, should the MMI generated for the household cleaner meet or exceed the threshold MMI, it may be determined that the household cleaner is likely to cause atopic dermatitis. Analogous decision-making can be completed for virtually any agent and condition of interest.

Determinations regarding the propensity of an agent to cause one or more conditions may be used to generate recommendations for agents, based upon, for example, determinations of MMIs. Recommendations may be based, for example, on ranked lists of agents or groupings of agents that may be generated based upon order of MMI values of an agent or of MMI values for each agent in a grouping of agents. Moreover, binary lists may also be generated such that lists of recommended and non-recommended agents or groupings of agents are compiled. Such lists may also include rankings within categories. Lists may be compiled such that recommended (or non-recommended) agents or groupings of agents are those that have MMI values that are at or above (or below) a given threshold value. Which side of a threshold that demarcates a recommended or non-recommended category may depend on the particular microbial communities that are altered when the host is exposed to an agent or groupings of agents of interest. For example, it may be that an agent or grouping of agents does not substantially alters the population of bacteria such that calculated MMI values are low, leading to the development of a condition. In such a case, an agent or grouping of agents may not be recommended. In other cases, it may be that an agent or grouping of agents substantially alters the population of bacteria such that calculated MMI values are high, leading to the development of a condition. In such a case, an agent or grouping of agents may also not be recommended.

Moreover, calculated MMI values may be used with other assays to determine whether or not bacterial modulation by an agent is harmful or beneficial. Such information can give insight as to whether an MMI generated for an agent represents a harmful or beneficial change in the abundance or function of microbial taxa. Non-limiting examples of assays that may used to determine whether or not bacterial modulation is harmful of beneficial include a blood assay, a urine assay, a fecal assay, a cerebrospinal fluid assay, a saliva assay, a sputum assay, an assay performed on a biopsy, an assay performed on part of the reproductive system, a cardiovascular assay, a respiratory assay, a cognitive assay, a reproductive assay, a liver function assay, a kidney function assay, a thyroid assay, a locomotor assay, an ocular assay, and combinations thereof. For example, an MMI may be calculated for an agent in parallel with a liver function assay conducted after administration of an agent to the subject. The results of both assays may be used to determine whether or not an agent is, for example, efficacious and/or safe.

MMI values may be used to provide health counseling services to one or more subjects in want or need. In general, health counseling generally comprises the steps of (a) identifying a subject in want or need of an agent; (b) obtaining an MMI for the agent(s); and (c) providing counseling to the subject regarding the agent based on the MMI. Counseling may be based off of MMI values obtained from samples from a subject, samples from subjects of the same species of the subject seeking counseling, or may be obtained from samples from subjects of a different species than of the subject seeking counseling. For example, human MMI values may be used to counsel a human subject. Alternatively, murine MMI values may be used to counsel a human subject. For example, MMI values may be estimated for a human subject from MMI values determined in subjects of a murine species. The estimated MMI values for a human subject may then be used to provide counseling. In another example, counseling may be provided using MMI estimates for one species of dog that are derived from MMI values generated in another species of dog.

Health counseling services may include deciding on a treatment regimen for a subject with a condition. In some examples, counseling may include deciding between two or more drugs available for treatment of the condition. MMIs may be obtained for available drugs from which to choose and used to determine which drug(s) and/or the dosage of drug(s) should be used for treatment.

For example, a patient with a condition is identified as in need of a therapy for the condition. Three different drugs are available to treat the condition, each drug known to exert its effects via microbial modulation. Using samples obtained from the patient, an MMI is generated for each agent using a calculation that includes condition importance weights (e.g., with respect to the patient's condition) for the enumerated microbial taxa. The generated MMI values are evaluated with respect to efficacy and safety. The drug with the most preferred MMI is selected for treatment. The MMI study can be repeated for different doses of the selected drug to determine an optimal dose of the selected agent.

In some examples, counseling may include advice for pursuing fecal transplants. Transplants may be initiated and MMIs calculated pre- and post-transplant in order to assess the utility of the fecal transplant.

Health counseling services may include the communication of a variety of pieces of information with respect to use of an agent, including recommendations. Non-limiting examples of such information includes information regarding the safety of an agent, information regarding the efficacy of an agent, information regarding the safety of an agent when administered with one or more different agents, information regarding the efficacy of an agent when administered with one or more different agents, a recommendation to use or continue to use an agent or combination of agents, a recommendation to not use or discontinue use of an agent or combination of agents, providing a ranked list of possible agents or combination of agents for use or continued use, recommendations for the addition of one or more different agents to a regimen comprising an agent or combination of agents, recommendations for monitoring use of an agent over time, recommendations for doses (including dosing frequency and dosing level) of an agent, recommendations for exposure (or avoiding exposure) to an agent, or combinations thereof.

Health counseling services, health decision-making, or other investigation may include the generation of one or more reports and such reports may be used as a part of health counseling. Such reports may be given to a subject in want or need in hard-copy form or may be transmitted electronically (e.g., via a computer network such as the Internet, a local computer network, via a display (e.g., a display with a graphical user interface), via email, etc.), or other electronic means. Reports may include raw data obtained from detecting microbial taxa, enumerations of microbial taxa abundance and/or functionality, a generated MMI, changes in abundance of one or more microbial taxa enumerated to generate an MMI, the algorithm used to generate an MMI, any appropriate statistics, information on how to interpret an MMI, and may also include summaries of provided counseling, including any of the various exemplary pieces of information and recommendations described above. Health counseling and/or a report may be provided by virtually anyone or any organization, including health care professionals (e.g., a physician, a nurse, a nurse practitioner, a physician's assistant, a nutritionist), a health care organization, a pharmaceutical company, and combinations thereof. A report may be provided to virtually anyone, including a subject from samples were obtained to determine an MMI, another subject, a subject seeking counseling, a physician, a nurse, a pharmaceutical company, an insurance company, and combinations thereof.

Moreover, any of the steps of the methods described herein may be completed via a computer network, such as, for example the Internet. For example, an MMI may be generated with the aid of information (e.g., variability weights, prevalence weights, condition importance weights, raw data used for enumerations) transmitted or received over the Internet. Moreover, a calculated MMI may be transmitted or received or the Internet.

### Systems for Determining and Interpreting a Microbiome Modulation Index

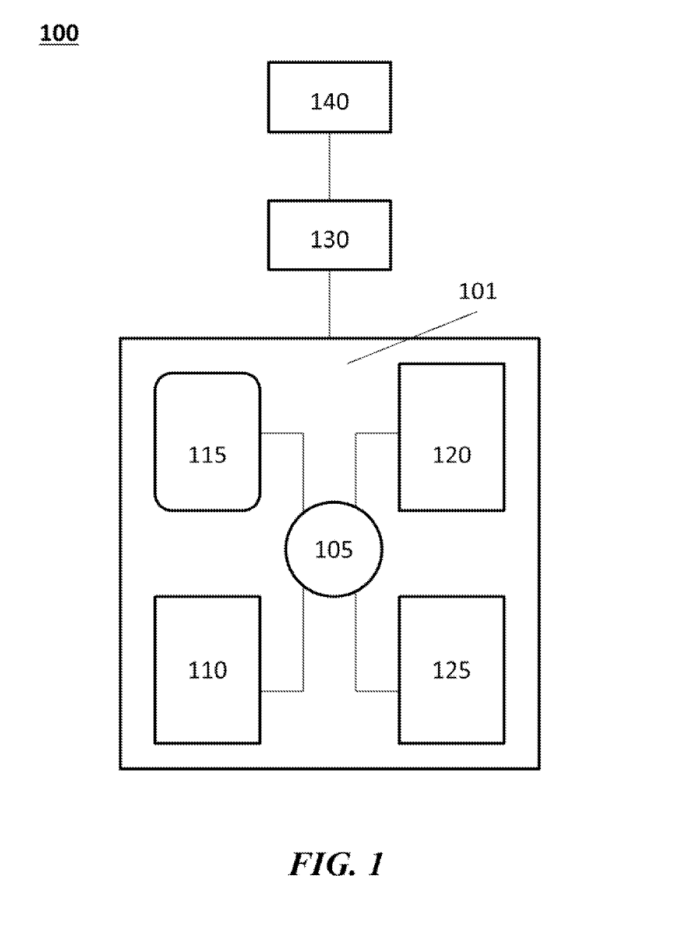

The disclosure provides specialized computer systems that are configured to implement methods of the disclosure, including the determination of an MMI and/or the interpretation of an MMI. Specialized computer systems are generally capable of any of the following: (a) accepting raw data (e.g., data for the detection of microbial taxa, data for the detection of chemical species related to a microbial taxa (e.g., nucleic acid)) that can be used to enumerate microbial taxa and related chemical species and calculate an MMI; (b) pre-processing the raw data such that it is acceptable for entry into MMI calculation algorithms; (c) calculating an MMI from the raw data or processed raw data; (d) outputting the MMI to a user; and (e) interpreting, in whole or part, the calculated MMI. In some cases, one or more of these capabilities may be performed using instructions received or transmitted over the internet. In some cases, a specialized computer system may also be capable of organizing microbial species into microbial taxa (e.g., OTUs), using, for example, nucleic acid sequences stored in a database. The system may include a computer server (“server”) that is programmed to implement the methods described herein. FIG. 1 depicts a system 100 adapted to receive raw data from detecting microbial taxa in samples; process the raw data obtained from detecting microbial taxa (e.g., enumerate the detected microbial taxa or related chemical species); calculate an MMI from the enumerated microbial taxa or related chemical species; output the MMI to a user; and/or interpret the generated MMI. The system 100 includes a central computer server 101 that is programmed to implement exemplary methods described herein. The server 101 includes a central processing unit (CPU, also “processor”) 105 which can be a single core processor, a multi core processor, or plurality of processors for parallel processing. In some cases, methods described herein can be executed with the aid of the processor. The server 101 also includes memory 110 (e.g. random access memory, read-only memory, flash memory); electronic storage unit 115 (e.g. hard disk); communications interface 120 (e.g. network adaptor) for communicating with one or more other systems; and peripheral devices 125 which may include cache, other memory, data storage, and/or electronic display adaptors. The memory 110, storage unit 115, interface 120, and peripheral devices 125 are in communication with the processor 105 through a communications bus (solid lines), such as a motherboard. The storage unit 115 can be a data storage unit for storing data. The server 101 may be operatively coupled to a computer network (“network”) 130 with the aid of the communications interface 120. The computer system may transmit or receive data of the computer network. The network 130 can be the Internet, an intranet and/or an extranet, an intranet and/or extranet that is in communication with the Internet, a telecommunication or data network. The network 130 in some cases, with the aid of the server 101, can implement a peer-to-peer network, which may enable devices coupled to the server 101 to behave as a client or a server.

The storage unit 115 can store files, such as raw data files from detecting microbial taxa, databases comprising prevalence weights, databases comprising variability weights, databases comprising condition importance weights, databases comprising determined MMIs, databases comprising reference MMIs, databases comprising nucleic acid sequences used to enumerate microbial taxa, databases comprising microbial taxa classification schemes, databases comprising microbial taxa or related chemical species, instructions to execute MMI calculation algorithms, databases of MMI calculation algorithms, interpretations (e.g., reports, input notes, etc.) of MMIs, combinations thereof, or any aspect of data associated with the executing methods of the disclosure.

The server can communicate with one or more remote computer systems through the network 130. The one or more remote computer systems may be, for example, personal computers, laptops, tablets, telephones, Smart phones, or personal digital assistants. Moreover, system 100 may be capable of accepting instructions over network 130 from one or more remote computer systems such that its data is accessed to calculate an MMI (either by the remote computer systems or system 100). Alternatively, system 100 is capable of accepting data stored, analyzed, and/or interpreted on a remote system that is transmitted to system 100 over network 130. Moreover, system 100 is also capable of transmitting data stored, analyzed, and/or interpreted by system 100 to one or more remote computers over network 130.

In some situations the system 100 includes a single server 101. In other situations, the system includes multiple servers in communication with one another through an intranet, extranet and/or the Internet.

The server 101 can be adapted to store raw data files from detecting microbial taxa, databases comprising prevalence weights, databases comprising variability weights, databases comprising condition importance weights, databases comprising determined MMIs, databases comprising reference MMIs, databases comprising nucleic acid sequences used to enumerate microbial taxa, databases comprising microbial taxa classification schemes, databases comprising microbial taxa or related chemical species, instructions to execute MMI calculation algorithms, databases of MMI calculation algorithms, interpretations (e.g., reports, input notes, etc.) of MMIs, combinations thereof, or any other aspect of data associated with the executing methods described herein. Such information can be stored on the storage unit 115 or the server 101 and such data can be transmitted through a network, such as network 130.

Methods as described herein can be implemented by way of machine (or computer processor) executable code (or software) stored on an electronic storage location of the server 101, such as, for example, on the memory 110, or electronic storage unit 115. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110. Alternatively, the code can be executed on a second computer system 140.

Aspects of the systems and methods provided herein, such as the server 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g. read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, tangible storage medium, a carrier wave medium, or physical transmission medium. Non-volatile storage media can include, for example, optical or magnetic disks, such as any of the storage devices in any computer(s) or the like, such may be used to implement the system. Tangible transmission media can include: coaxial cables, copper wires, and fiber optics (including the wires that comprise a bus within a computer system). Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, DVD-ROM, any other optical medium, punch cards, paper tame, any other physical storage medium with patterns of holes, a RAM, a ROM, a PROM and EPROM, a FLASH-EPROM, any other memory chip or cartridge, a carrier wave transporting data or instructions, cables, or links transporting such carrier wave, or any other medium from which a computer may read programming code and/or data. Many of these forms of computer readable media may be involved in carrying one or more sequences of one or more instructions to a processor for execution.

Interpretation of an MMI can include the generation of one or more reports, including any of the types of reports described herein. In some examples, the report(s) or output of an MMI may be presented to a user with the aid of a user interface, such as an electronic display of a system that may comprise a graphical user interface (GUI). In some examples, systems may include a printer device (not shown in FIG. 1) that is capable of producing paper hard copies of any information displayed to a user or may simply provide the report in hard copy form without a coupled electronic display. Non-limiting examples of paper hard copies that may be generated by the printer include reports that summarize the calculation of an MMI, interpretations of the MMI, recommendations based on the calculation of MMI, and/or producing ranked lists of agents or groupings of agents based on MMI.

Moreover, a specialized computer system may also aid in providing health counseling and/or making health decisions. For example, a specialized computer system may be capable of generating an MMI from data obtained from patient samples, may be capable of storing patient records, may be capable of generating a report based on a generated MMI, may be capable of communicating with a patient electronically (e.g., via the internet, via email), may be capable of providing a summary of counseling provided to a subject based on an MMI, and combinations thereof. In some cases, reports generated by a specialized computer system may be used in providing health counseling to a subject and/or to make health decisions described elsewhere herein.

### EXAMPLES

### Example 1

### Calculation of an MMI

An agent is administered to a group of mice in a standard controlled experiment design. The constituent taxa of microbial communities are enumerated in fecal samples obtained before and after administration of the mice with the agent. An MMI estimate for the agent in human is then determined using the enumerations from the murine samples.

Adult mice are treated with the agent of interest via oral gavage. The mice are individually caged (important because mice consume stool of other mice, which transfers some microbes across mice). Stool samples are collected from the mice both before and after treatment, and those stool samples are used to assess the microbial communities present in the stool (and by proxy, the mouse gut).

Enumeration of the microbes in a community of interest is performed with high-throughput sequencing of the 16S rRNA gene (see e.g., Yatsunenko et. al, Nature 486, 222-227 (14 Jun. 2012), Kuczynski et al., Nature Reviews Genetics 13, 47-58 (January 2012), which are incorporated entirely herein by reference) although other methods can be applied. The resulting data reveals the abundance of each microbial taxon in each sample. Microbial taxa are organized as operational taxonomic units (OTUs), although other taxonomic classification schemes may also be used.

To calculate the MMI of a substance, an intermediate value d is calculated for each mouse administered with the agent. The abundance of each OTU before treatment in that mouse is compared to the abundance of that OTU after treatment using exemplary Equation 8:

$$d = \frac{{Σ}_{i} {f}_{i} * {g}_{i} * \vert {A}_{1 i} - {A}_{0 i} \vert}{{Σ}_{i} {A}_{1 i} + {A}_{0 i}} \qquad ( 8 )$$

where A₁ᵢ represents the abundance of OTU i at time-point 1 (post-administration),
where A₀ᵢ represents the abundance of OTU i at time-point 0 (pre-administration)
and fᵢ and gᵢ represent weights applied to each OTU, discussed below.

Once d is calculated for each mouse, d values for each administered mouse are averaged, and the resulting d̅ is compared to a reference level of change d₀ in a control group of mice not administered with the agent. The value d₀ is determined on the basis of laboratory studies of untreated mice in similar conditions.

The MMI is then calculated as in exemplary Equation 9:

MMI= d̅/d₀ (9)

for each substance of interest.

fi is a weight applied to each OTU based on its prevalence in human communities. Using a group of stool samples from humans, the abundance of various OTUs is observed in those human samples. fi is then the fraction of human samples where the OTU was present. OTU presence may be determined by any suitable means known in the art (see e.g., Hazen et. al, Science 330(6001), 204-208 (8 Oct. 2010), which is incorporated entirely herein by reference). In some cases, presence of an OTU may be determined by detecting any sequence associated with OTU. In some cases, presence of an OTU may be determined as a fraction of the sample that comprises a sequence associated with an OTU (e.g., greater than 1 sequence belonging to the OTU per 1 million sequences in the sample).

fi=1 implies that an OTU was found in all human samples and fi=0 implies that the OTU was found in none of the human samples.

gᵢ is a weight applied to each OTU based on its variability in untreated mice. Using a group of samples from untreated mice (e.g., a similar number of samples to the number of samples obtained from treated mice), the relative abundance of that OTU is measured across a similar period of time used in treated mice. The mean of the absolute value of the difference in relative abundance of that OTU in the samples from the untreated mice (δ), forms the basis for gᵢ as in exemplary Equation 10:

gᵢ=1−δ/δₘₐₓ (10)

where δₘₐₓ is the OTU with the largest delta in the untreated mice.

gᵢ=0 for the most variable OTU, and gᵢ=1 for OTUs which do not change in relative abundance over time in untreated mice.

### Example 2

### Calculation of MMIs for a Panel of Agents

### Mouse Multi-Dosing

Twenty five agents were selected to span multiple indications (Table 1). For each agent, the effective dose for the mouse was determined through a comprehensive search of the literature (Table 1). Working formulations were created by suspending agents in 1× phosphate buffered saline (PBS).

A total of 156 six-week old c57bl/6 mice (78 males and 78 females) were housed in separate cages 5 days prior to agent administration. Animals were handled daily by caretakers during this acclimation period. Animals were fed a standard chow diet throughout the course of the study.

Animals were treated with agents via oral gavage daily for 5 consecutive days beginning on day 1. N=6 mice per agent; 3 male+3 female. Fecal samples were collected using the ‘clean catch’ method in which the mice were held by the tail, causing them to defecate. Feces were collected in a sterile tube and immediately frozen and stored at −80° C. Fecal samples were collected in the morning prior to agent administration on the following days: −1,0,4,5.

### DNA Quantification and Amplification

Frozen day 0 and day 5 fecal specimens corresponding to 15 treatment groups (PBS+(Table 3) were profiled using Second Genome's Microbiome Signature Discovery™ service (San Bruno, Calif.). DNA was isolated using the PowerSoil DNA Isolation Kit following the manufacturer's instructions (MoBio Laboratories, Carlsbad, Calif.). For V4 sequencing assay profiling, the V4 region of the 16S rRNA genes was amplified using fusion primers tailed with Illumina sequencing adapters and indexing barcodes according to a previously described technique (Caporaso, 2010, PMID: 20534432). For each sample, amplified products were concentrated, purified and quantified by electrophoresis using an Agilent 2100 Bioanalyzer, then pooled for sequencing using the MiSeq (Illumina, San Diego, Calif.) instrument.

Using the software QIIME (Kuczynski, 2012, PMID: 23184592) and custom scripts, sequences were quality filtered and de-multiplexed using exact matches to the supplied DNA barcodes. Resulting sequences were then clustered into reference OTUs (rOTUs) by uclust (Edgar, 2010, PMID: 20709691) matching each sequence against the Greengenes sequences pre-clustered at 97%, a practice commonly referred to as “closed-reference OTU picking”. The longest Illumina-generated sequence from each of OTUs thus formed was then used as the OTU representative sequence, and assigned taxonomic classification via mothur's bayesian classifier (Schloss, 2009, PMID: 19801464) trained against the Greengenes database. Taxonomic annotations at 80% bootstrapped confidence were applied. Total sequence counts per fecal sample were scaled to be identical. Seven samples (MDO5M027D0, MD10M057D0, Md12F07D0, MD01F006D5, MD06F035D5, MD13M074D5, and MD04F023D5) were excluded following sequence quality filtering and alignment due to low sequence yield. Taxa were filtered to those present in at least one of the samples.

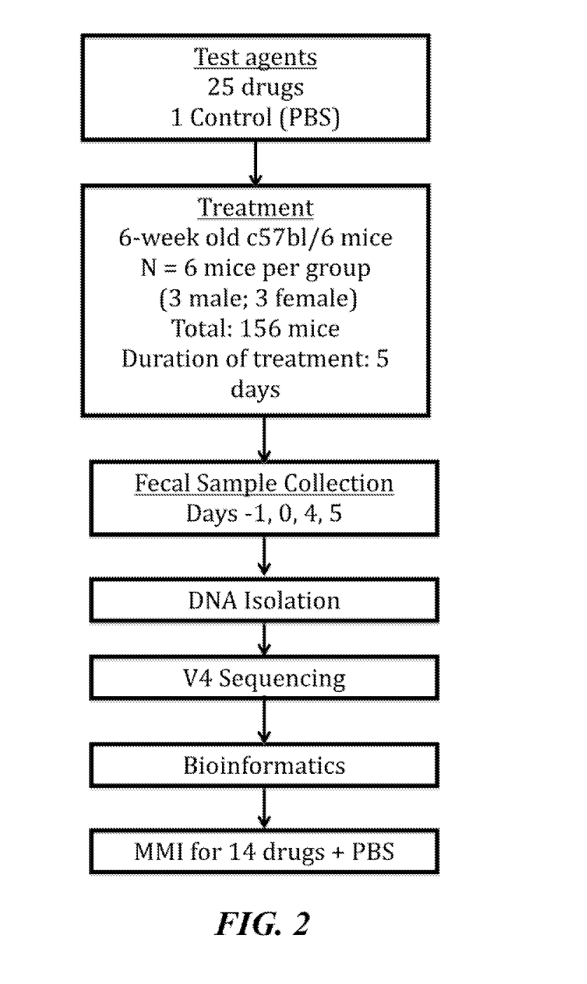

Experimental details are summarized in both Table 2 and in a flow-chart in FIG. 2.

### MMI and Sample-to-Sample Dissimilarity Functions

MMIs were calculated for the PBS control and 14 of the tested drugs. To calculate the MMI of an agent, an intermediate value d was calculated for each mouse administered with the respective agent. The abundance of each OTU before treatment in that mouse was compared to the abundance of that OTU after treatment as in exemplary Equation 11:

$$d = \frac{{Σ}_{i} {g}_{i} * \vert {A}_{1 i} - {A}_{0 i} \vert}{{Σ}_{i} ( {A}_{1 i} + {A}_{0 i} )} \qquad ( 11 )$$

where A₁ᵢ represents the abundance of OTU i at time-point 1 (post-administration),
where A₀ᵢ represents the abundance of OTU i at time-point 0 (pre-administration)
gᵢ represents a variability weight applied to each OTU, discussed below.

Once d was calculated for each mouse, d values for each administered mouse are averaged, and the resulting d̅ was compared to a reference level of change d₀ in a control group of mice not administered the agent. The value d₀ is determined on the basis of laboratory studies of untreated mice in similar conditions (e.g., a similar number of samples to the number of samples obtained from treated mice).

The MMI is then calculated as in exemplary Equation 12:

MMI= d̅/d₀ (12)

for each substance of interest.

gᵢ is a weight applied to each OTU based on its variability in untreated mice. Using a group of samples from untreated mice (e.g., a similar number of samples to the number of samples obtained from treated mice), the relative abundance of that OTU is measured across a similar period of time used in treated mice. The mean of the absolute value of the difference in relative abundance of that OTU in the untreated mice (δ), forms the basis for gᵢ as in exemplary Equation 13:

gᵢ=1−δ/δₘₐₓ (13)

where δₘₐₓ is the OTU with the largest delta in the untreated mice.

gᵢ=0 for the most variable OTU, and gᵢ=1 for OTUs which do not change in relative abundance over time in untreated mice.

All profiles were inter-compared in a pair-wise fashion to determine the MMI for each treatment group. Significance testing of the weighted Unifrac and Unifrac community dissimilarity pre/post-treatment was determined with the ANOVA test. Statistical significance was concluded in cases where a p-value was p<0.05. The results of significance testing are shown in Table 3 with calculated MMI values. Statistical significance is indicated with an asterisk.

The calculated MMIs are then stored in a database on a specialized computer system, accessible to health care professionals. The stored MMIs may be used to provide health counseling to subjects.

TABLE 1
List of agents tested in experiments of Example 2

| | | Agent | | | |
|---|---|---|---|---|---|
| | | (Compound) | | | |
| Agent | Indication | Name | Mouse Dose | | References |
| Pentasa | IBD | Mesalamine | 75 | mg/kg p.o. | http://clincancerres.aacrjournals.org/content/13/21/6527.long |
| Ritalin | ADHD | Methylphenidate | 5 | mg/kg p.o. | Brain Research Volume 1357, |
| | | hydrochloride | | | 21 Oct. 2010, Pages 62 [symbol] 69 |
| Viagra | Erectile | Sildenafil | 50 | mg/kg p.o. | Cytokine. 2012 Nov; 60(2): 540-51 |
| | dysfunction | | | | |
| Precose | T2D | Acarbose | 40 | mg/kg | Metabolism. 2001 |
| | | | | | Sep; 50(9): 1049-53 |
| Prozac | Depression | Fluoxetine | 10 | mg/kg | J Neurochem. 2012 Sep 26 |
| | | hydrochloride | | | |
| Fosamax | Osteoporosis | Alendronate | 0.1 | mg/kg | J Bone Miner Res. 2009 |
| | | sodium | | | Feb; 24(2): 196-208 |
| | | trihydrate | | | |
| Nexium | GERD | Esomeprazole | 20 | mg/kg p.o. | Astudillo L, Rodriguez JA, et al., |
| | | magnesium | | | J Pharm Pharmacol. 2002 |
| | | hydrate | | | Apr; 54(4): 583-8. |
| Metformin | Type 2 | Metformin | 400 | mg/kg p.o. | Toyama K, et al. Br J |
| | diabetes | | | | Pharmacol. 2012 |
| | | | | | Jun; 166(3): 1183-91.; |
| | | | | | Heishi M, et. al. Diabetologia. |
| | | | | | 2006 Jul; 49(7): 1647-55. |
| Amoxicilin | Infections | Amoxicillin | 200 | mg/kg p.o. | Clark J M, et. al. Antimicrob |
| | | | | | Agents Chemother. 1987 |
| | | | | | Feb; 31(2): 226-9. |
| Azithromycin | Infections | Azithromycin | 50 | mg/kg p/o/ | Girard A E, et al. Antimicrob |
| | | | | | Agents Chemother. 1987 |
| | | | | | Dec; 31(12): 1948-54 |
| Metranidazole | Infections | Metranidazole | 250 | mg/kg p.o. | Reznikov M, et al. |
| | | | | | Chemotherapy. 1985; 31(1): 50-4. |
| Seroquel | Antipsychotic | Quetiapine | 30 | mg/kg p.o. | Pisu C, et al. Behav Pharmacol. |
| | | hemifumarate | 100 | mg/kg p.o. | 2010 Oct; 21(7): 649-53. |
| | | salt | | | Egashira N, et al. Eur J |
| | | | | | Pharmacol. 2008 Sep |
| | | | | | 11; 592(1-3): 103-8. |
| Aricept | Alzheimer's | Donepezil | 1.0 | mg/kg p.o. | Freret T, et al. Behav Brain |
| | Disease | | | | Res. 2012 Apr 21; 230(1): 304-8.; |
| | | | | | Furukawa-Hibi Y, et al. |
| | | | | | Behav Brain Res. 2011 Nov |
| | | | | | 20; 225(1): 222-9.; Riedel G, et |
| | | | | | al. Behav Brain Res. 2009 Dec |
| | | | | | 1; 204(1): 217-25. |
| Lipitor | Cholesterol | Atorvastatin | 40 | mg/kg p.o. | Paraskevas Klet al. Angiology. |
| | lowering | calcium salt | | | 2011 Feb; 62(2): 144-54. |
| | | trihydrate | | | |
| Plavix | ACS, MI, | Clopidogrel | 20 | mg/kg p.o. | Abele Set al. Transplantation. |
| | stroke | hydrogensulfate | | | 2009 Jan 27; 87(2): 207-16. |
| Estrogen | Hot flashes | 17-beta | 0.18 | mg/kg p.o. | Fernandez SM, et al. Behav |
| | | estradiol | | | Neurosci. 2004 |
| | | | | | Dec; 118(6): 1340-51. |
| Linosipril | Hypertension | Lisinopril | 10 | mg/kg p.o. | Rousseau-Plasse A, et al. Exp |
| | | | | | Hematol. 1998 |
| | | | | | Oct; 26(11): 1074-9. |
| Prednisalone | | Prednisalone | 10 | mg/kg | Proc Natl Acad Sci USA. 2004 |
| | | | | | Nov 2; 101(44): 15736-41 |
| Advil | Pain relief | Ibuprofen | 50-80 | mg/kg | web.jhu.edu/animalcare/procedures/mouse.html |
| | | sodium salt | | | |
| Aspirin | Pain relief | Acetylsiacylic | 400 | mg/kg | web.jhu.edu/animalcare/procedures/mouse.html |
| | | acid | | | |
| Benadryl | Allergies/cold | Diphenhydramine | 20 | mg/kg | Int Arch Allergy Immunol. |
| | | hydrochloride | | | 2011; 155(4): 355-60. |
| Caffeine | Stimulant | Caffeine | 20 | mg/kg | Psychopharmacology (1999) |
| | | | | | 144: 61 [symbol] 66. Can be |
| | | | | | teratogenic at 100 mg/kg: |
| | | | | | Hum Exp Toxicol 1981 1: 53 |
| Diflucan | Anti-fungal | Fluconazole | 50 | mg/kg p.o. | Majithiya J, et al.. J Antimicrob |
| | | | | | Chemother. 2009 |
| | | | | | Jan; 63(1): 161-6. |
| | | | | | Kamberi P, et al. 2007 |
| | | | | | Mar; 51(3): 998-1003. |
| Singulair | Asthma, | Montelukast | 0.5 | mg/kg | FASEB J. 2007 |
| | allergies | sodium hydrate | | | Dec; 21(14): 3877-84 |
| Actos | T2D | Pioglitazone | 30 | mg/kg p.o. | Mohapatra J, et al. |
| | | hydrochloride | | | 2009; 84(4): 203-10. |

TABLE 2
Summary of experiments in Example 2

| | | | | Total |
|---|---|---|---|---|
| | | | | number of |
| | | | | fecal |
| Design | Drugs | Total Mice | Time-points | samples |
| 6 mice × 26 test | (neg control, | 156 | −1, 0, 4, 5 | 624 |
| agents (1 PBS | 25 secondary | | | |
| control + 25 | standard | | | |
| drugs) | compounds | | | |
| 3 male mice and | spanning | | | |
| 3 female mice | multiple | | | |
| per group | indications | | | |
| | (Table 1)) | | | |

TABLE 3
Calculated MMIs for select drugs evaluated in Example 2

| | | | | | p-value | |
|---|---|---|---|---|---|---|
| | | Group | | MMI Standard | (W- | p-value |
| Drug | Brand Name | number | MMI | deviation | unifrac) | (Unifrac) |
| PBS | PBS | MD01 | 1 | 0.241931695 | 0.026* | 0.031* |
| Fluconazole | Diflucan | MD06 | 1.00342796 | 0.172326869 | 0.026* | 0.046 |
| Acetylsiacylic_acid | Aspirin | MD14 | 1.093072705 | 0.345887067 | 0.802 | 0.069 |
| Esomeprazole_magnesium_hydrate | Nexium | MD04 | 1.099936623 | 0.194439825 | 0.066 | 0.690 |
| Alendronate_sodium_trihydrate | Fosamax | MD13 | 1.10743671 | 0.257740601 | 0.330 | 0.234 |
| Mesalamine | Mesalamine | MD02 | 1.130791046 | 0.330071827 | 0.051* | 0.033* |
| Diphenhydramine_hydrochloride | Benadryl | MD15 | 1.201665933 | 0.272736882 | 0.156 | 0.039* |
| Dicyloamine_hydrochloride | Bentyl | MD03 | 1.221961838 | 0.25289047 | 0.006* | 0.001* |
| Prednisalone_21- | Prednisalone | MD07 | 1.261711241 | 0.20410056 | 0.028* | 0.054 |
| hemisuccinate_sodium_salt | | | | | | |
| Atorvastatin_calcium_salt_trihydrate | Lipitor | MD12 | 1.277992735 | 0.636326951 | 0.464 | 0.256 |
| Fluoxetine_hydrochloride | Prozac | MD10 | 1.439805816 | 0.179537122 | 0.008* | 0.014* |
| Zolpidem_tartrate | Ambien | MD11 | 1.442529481 | 0.308353789 | 0.040* | 0.044* |
| Metformin_hydrochloride | Metformin | MD05 | 1.628410308 | 0.291494989 | 0.068 | 0.008* |
| Azithromycin | Zithromax | MD09 | 2.20193981 | 0.141045582 | 0.010* | 0.002* |
| Amoxycillin | Amoxil | MD08 | 2.396675612 | 0.005256015 | 0.002* | 0.002* |

*= statistical significance

### Example 3

### Using an MMI to Make a Health Decision Regarding a Drug

A drug is administered to a group of mice in a standard controlled experiment design. The constituent taxa of microbial communities are enumerated in fecal samples obtained from the mice before and after administration of the mice with the drug. An MMI estimate for the drug in humans is then determined using the murine enumerations and the MMI algorithm described in Example 1. The MMI estimate generated for the drug in humans is stored in a database accessible by a health care provider, who determines that the MMI generated mice represents beneficial modulation of microbial taxa. The health care provider identifies a subject in want or need of the drug and decides to treat the subject with the drug based on the MMI estimate in humans generated using enumerations in mice.

### Example 4

### Using an MMI to Make a Health Decision Regarding a Drug

A drug is administered continuously to a livestock with a condition. Prior to and after each administration of the drug, fecal samples from the livestock are obtained. Related chemical species (e.g., nucleic acids, V4 region of 16S rRNA, etc.) to relevant microbial taxa of interest are enumerated to generate an MMI for the agent after each administration. After several administrations of the drug, the MMI remains unchanged at an undesirable level. Comparison of the MMIs with each administration of the agent is used to decide to cease treatment of the livestock with the drug. Another drug is administered to the livestock and fecal samples obtained and related chemical species enumerated as above. A desirable MMI is generated and used to decide to continue treatment of the livestock with the second drug.

### Example 5

### Using an MMI to Make a Health Decision Regarding a Bandage

A company has invented a new bandage considered for human use. The bandage is applied to the back sides of a group of mice in a standard controlled experiment design. Where the bandage has been applied, the mouse's fur has been removed. The constituent taxa of microbial communities are enumerated in skin samples obtained from the mice before and after application of the bandages to the mice. An MMI estimate for the bandages in humans is then determined using the murine enumerations and the MMI algorithm described in Example 1. It is determined that a favorable MMI estimate in humans is obtained by comparing the MMI obtained for the new bandage to MMIs obtained for humans for bandages already approved for human use. Based on the favorable MMI estimate in humans, a decision is made to further test the new bandage on human subjects.

### Example 6

### Providing Health Counseling Based on an MMI

A subject is in want of a particular diet. The subject visits with a nutritionist who has access to a database of MMI values for various diets. Upon visiting the nutritionist, the nutritionist accesses a database of human MMI values for diets and locates an MMI value for the diet (e.g., an average MMI generated from the individual MMIs of the component foods in the diet) the subject is interested in. The database comprises MMI values for different age groups of subjects and also provides recommendations (e.g., via a list of recommended and non-recommended diets) for various age groups. The nutritionist notes that, based on the MMI value for diet for subjects of the subject's age group, the diet is not recommended. The nutritionist then counsels the subject not to undertake the diet and provides the subject with the MMI value for the diet, information on how to interpret the MMI value, along with other recommended diets with more acceptable MMIs for the subjects of the subject's age range.

### Example 7

### Providing Health Counseling Based on an MMI

A human subject is interested in starting a new vitamin regimen, but is concerned that the vitamin may modulate microbiota associated with the development of a condition. Prior to and after administration of the vitamin to the subject, fecal samples are obtained from the subject and a panel of microbial taxa are enumerated. From these enumerations an MMI is generated by a specialized computer system using an algorithm with condition weights, such that microbial taxa critical to the condition of interest are weighted accordingly in the MMI calculation.

The calculated MMI is stored in the specialized computer system and compared with MMIs calculated for the vitamin in other human subjects who were known to have developed the condition after taking the vitamin and subsequently stored in a database on the specialized computer system. The specialized computer system compares the vitamin's calculated MMI in the subject with other MMIs in the database and determines that the agent has a substantially more favorable MMI in the subject than those in the database known to have developed the condition. The specialized computer system generates a report displayed on its electronic display. A physician evaluating the agent in the subject discusses the report with the subject and counsels the subject to commence the vitamin regimen. A copy of the report is also transmitted over the internet to the subject's email address such that the subject can review the report at a later time.

It should be understood from the foregoing that, while particular implementations have been illustrated and described, various modifications may be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

1. A method for characterizing an agent comprising:
a. enumerating abundance of one or more microbial taxa or related chemical species in one or more first samples obtained from a first subject prior to administering the agent to the subject;
b. administering the agent to the first subject;
c. enumerating abundance of the one or more microbial taxa or related chemical species in one or more second samples obtained from the first subject after the administering the agent to the first subject;
d. generating an index for the agent using:
i. the enumerating abundance of the one or more microbial taxa or related chemical species in one or more first samples obtained from a first subject prior to administering the agent to the subject in step (a);
ii. the enumerating abundance of the one or more microbial taxa or related chemical species in one or more second samples obtained from the first subject after the administering the agent to the first subject in step (b); and
iii. at least one of a prevalence weight, a variability weight, or a condition importance weight.

2. The method of claim 1, further comprising comparing the index with one or more reference indices, wherein the comparing is used to make a health decision with respect to the agent.

3. The method of claim 1, wherein the variability weight is generated using the relative abundance variability of each of the one or more microbial taxa or related chemical species in samples obtained from the first subject, wherein the agent has not been administered to the first subject.

4. The method of claim 1, wherein the prevalence weight is calculated using the relative abundance of each of the microbial taxa or related chemical species in third samples obtained from a second subject.

5. The method of claim 4, wherein the first subject and the second subject are of a different species.

6. (canceled)

7. The method of claim 1, wherein the condition importance weight is with respect to a condition of interest selected from the group consisting of Clostridium difficile infection, inflammatory bowel disease (IBD), a condition of the gut, Crohn's Disease (CD), irritable bowel syndrome (IBS), stomach ulcers, colitis, neonatal necrotizing enterocolitis, gastroesophageal reflux disease (GERD), gastroparesis, cystic fibrosis, chronic obstructive pulmonary disease, rhinitis, atopy, asthma, acne, a food allergy, obesity, periodontal disease, diarrhea, constipation, functional bloating, gastritis, lactose intolerance, visceral hyperalgesia, colic, pouchitis, diverticulitis, allergies, asthma, sinusitis, chronic obstructive pulmonary disorder (COPD), depression, attention deficit hyperactivity disorder (ADHD), autism, Alzheimers, migraines, multiple sclerosis (MS), Lupus, arthritis, Type 2 diabetes, obesity, non alcoholic steato hepatitis (NASH), non alcoholic fatty liver disease (NAFLD), risk of infarction/cardiovascular risk, heart failure, cancer, dental caries, gingivitis, oral cancer, oral mucositis, bacterial vaginosis, fertility, sinusitis, allergies, cystic fibrosis, lung cancer, psoriasis, atopic dermatitis, methicillin-resistant staphylococcus aureus (MRSA), and combinations thereof.

8. The method of claim 1, wherein a plurality of first subjects are administered the agent, wherein the one or more first samples are obtained from each first subject of the plurality prior to the administering, wherein the one or more second samples are obtained from each first subject of the plurality after the administering, and wherein the index is calculated using the equation:
index= d̅/d₀
wherein is the average value d calculated for each first subject in the plurality,
wherein d is calculated using the equation:
$$d = \frac{{Σ}_{i} {g}_{i} * {f}_{i} * {h}_{i} * \vert {A}_{Ti} - {A}_{0 i} \vert}{{Σ}_{i} {A}_{1 i} + {A}_{0 i}}$$
wherein gᵢ is the variability weight,
wherein fᵢ is the prevalence weight,
wherein hᵢ is the condition importance weight,
wherein A_{Ti} is the abundance of the microbial taxa or related chemical species i in the one or more second samples obtained at time T after the administering, and
wherein Aₒₙ is the abundance of the microbial taxa or related chemical species i in the one or more first samples obtained prior to the administering; and
wherein d₀ is the average value d calculated for a plurality of third samples obtained from one or more second subjects identical to the first subjects but not administered the agent.

9.-11. (canceled)

12. The method of claim 2, wherein the health decision is made for at least one second subject that is of a different type of living organism than the first subject.

13.-15. (canceled)

16. The method of claim 1, wherein the microbial taxa are operational taxonomic units (OTUs).

17. The method of claim 16, wherein the OTUs are formed by clustering nucleic acid sequences of microbial organisms based on gene sequence homology.

18. The method of claim 17, wherein the OTUs are characterized by microbes having at least 80%, at least 85%, at least 90%, or at least 95% 16S RNA sequence homology.

19. (canceled)

20. The method of claim 1, wherein the one or more first samples and the one or more second samples are obtained from at least one of the following: the gut, the vagina, the cervix, the respiratory system, the ear, nasal passages, an oral cavity, a sinus, a nare, the urogenital tract, skin, feces, udders, auditory canal, earwax, breast milk, blood, sputum, urine, saliva, open wounds, secretions from open wounds, and combinations thereof.

21. The method of claim 20, wherein the one or more first samples and the one or more second samples are obtained from feces.

22. The method of claim 1, wherein the agent is selected from the group consisting of a microbe, a virus, a prebiotic, a probiotic, a synbiotic, a fecal transplant, a small molecule drug, a biologic drug, an orally administered drug, a parenterally administered drug, an antibiotic, a food, a beverage, a nutraceutical, a supplement, a beauty care product, personal hygiene product, an allergen, a household chemical, a wound dressing, a wound antiseptic, an industrial chemical, a hazardous chemical, water from a municipal water source, an environmental sample, an aerosol that may be inhaled via the nose or throat, a topical pain reliever, a material used to make clothing, and combinations thereof.

23. (canceled)

24. The method of claim 2, wherein the health decision is determining the safety and/or efficacy of the agent.

25. The method of claim 2, wherein two or more agents are administered in step (b) and wherein the health decision is determining the safety and/or efficacy of administering the two or more agents in combination.

26. The method of claim 2, wherein the health decision is deciding whether the agent can ameliorate the deleterious effects of one or more other agents on the one or more microbial taxa or related chemical species.

27.-31. (canceled)

32. The method of claim 2, wherein the agent is a drug and the health decision is determining a dose of the drug.

33. The method of claim 1, wherein the agent is a food, optionally a food of a diet.

34. The method of claim 2, wherein the health decision is determining whether the agent causes a condition.

35. (canceled)

36. The method of claim 1, wherein the enumerating the abundance of the one or more microbial taxa or related chemical species in step (a) and step (c) is completed by detecting a species selected from the group consisting of a nucleic acid, a lipid, a carbohydrate, a protein, a peptide, a small molecule, and combinations thereof.

37. (canceled)

38. The method of claim 1, wherein the enumerating the abundance of the one or more microbial taxa or related chemical species in step (a) and step (c) is completed by detecting all or a portion of a 16S ribosomal RNA (rRNA) gene or the 16S rRNA product of the gene.

39. (canceled)

42. A method of providing health counseling, comprising:
a. identifying a subject in want or need of an agent;
b. characterizing the agent according to the method of claim 1, thereby generating an index for the agent; and
c. providing counseling regarding the agent to the subject using the index.

43.-49. (canceled)

50. The method of claim 42, wherein the counseling includes any of the following: providing the subject with information regarding the efficacy of the agent; providing the subject with information regarding the safety of the agent; providing the subject with information regarding the safety of the agent when administered with one or more different agents; providing the subject with information regarding the efficacy of the agent when administered with one or more different agents; providing the subject with a recommendation to use or continue to use the agent or a combination of agents including the agent; providing the subject with a recommendation to not use or discontinue use of the agent or a combination of agents comprising the agent; providing the subject with a ranked list including the agent or a combination of agents comprising the agent for use or continued use; providing the subject with a recommendation for the addition of the agent to a regimen comprising one or more different agents; providing the subject with a recommendation for monitoring use of the agent over time; providing the subject with a recommended dose of the agent or a combination of agents comprising the agent; and combinations thereof.

51. (canceled)

52. A specialized computer system that is capable of performing the following:
a. accepting raw data that can be used to enumerate microbial taxa or related chemical species and characterize an agent according to the method of claim 1, thereby generating an index for the agent;
b. processing the raw data such that it may be used to calculate the index;
c. calculating the index; and
d. outputting the index to a user.

53.-57. (canceled)

58. The specialized computer system of claim 52, wherein the specialized computer system comprises any of the following databases: a database comprising reference indices, a database comprising nucleic acid sequences, a database comprising prevalence weights, a database comprising variability weights, a database of calculated indices, a database comprising microbial taxa classification schemes, a database comprising microbial taxa and/or related chemical species, and combinations thereof.
